(19) United States
(12) Patent Application Publication
(10) Pub. No.: US 2008/0249076 A1
(43) Pub. Date: Oct. 9, 2008

(54) Pharmaceutical Compositions Comprising Danazol

(75) Inventors: Per Holm, Vanlose (DK); Tomas Norling, Lyngby (DK)

Correspondence Address: DARBY & DARBY P.C., P.O. BOX 770, Church Street Station, New York, NY, 10008-0770, US

(73) Assignee: LifeCycle Pharma A/S

(21) Appl. No.: 10/581,170

(22) PCT Filed: Dec. 3, 2004
(86) PCT No.: PCT/DK04/00844

§ 371 Date: Sep. 19, 2006

(30) Foreign Application Priority Data
Dec. 3, 2003 (DK) PA 2003 01785

(51) Int. Cl. A61K 31/58

(52) U.S. Cl. 514/176

(57) ABSTRACT

A controlled release pharmaceutical comprising danazol has the property of slow release of danazol over an extended period of time and markedly increased bioavailability compared to commercially available danazol-containing products. The pharmaceutical composition comprises danazol dissolved in a solid vehicle or carrier and is especially suitable for oral solid dosage forms. The composition significantly reduces food effect and may reduce side effects.

### FIELD OF THE INVENTION

The present invention relates to a controlled release pharmaceutical composition that achieves slow release of danazol over an extended period of time and markedly increased bioavailability compared to commercially available danazol containing products. Furthermore, compositions according to the invention provide a significantly reduced food effect and are expected to reduce side effects.

In particular the invention relates to solid pharmaceutical compositions comprising danazol dissolved in a solid carrier formulated for oral administration.

### BACKGROUND OF THE INVENTION

Danazol is a synthetic steroid analog that has strong antigonadotropic properties. It is a synthetic androgen derived from ethisterone. It supresses the pituitary-ovarian axis by inhibiting the output of pituitary gonadotropins and depresses output of both follicle-stimulating hormones (FSH) and luteinizing hormone (LH). Danazol appears to exert its inhibitory effect by binding receptors of ganadal steroids at target organs. Danazol will decrease IgG, IgM and IgA levels, as well as phospholipid and IgG isotope auto antibodies. Danazol has been used in the treatment of endometriosis by altering the normal and ectopic endometrial tissue so that it becomes inactive and atrophic. Danazol is also prescribed for hereditary angioedema, fibrocystic breast disease, premenstrual syndrome, breast cancer and idiopathic thrombocytopenic purpura. Danazol is metabolized hepatically and undergoes significant first pass metabolism. Blood levels of danazol do not typically increase with increased oral doses.

Danazol comprises 17-alpha-pregna-2,4-dien-20-yno[2,3-d]isoxazol-17-ol as its chemical entity, the structural formula is

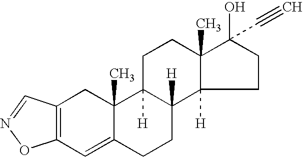

The molecular weight is about 337.

For treatment of moderate to severe endometriosis it is normally administered to women in dosages of up to 800 mg daily in two divided doses. At such higher doses, adverse side effects are seen which may include weight gain, virilism including voice change, development of facial and chest hair, loss of libido, acne, decreased bone mineral content and central nervous system symptoms such as depression, anxiety, fatigue, nausea and diarrhea, as well as inhibition of pregnancy while undergoing treatment. For mild endometriosis, the initial daily oral dose of 200 to 400 mg in two divided doses is recommended. It is imperative that treatment continues uninterrupted for 3 to 6 months, and may be extended if necessary. The total daily dose for fibrocystic breast disease is 100 to 400 mg given orally in two divided doses. The initial dose of danazol for the treatment of hereditary angioedema is 200 mg orally, 2 or 3 times daily.

The high doses of danazol are required because the bioavailability of commercially available danazol products is very low (about 10%). The low bioavailability is due to the low solubility of danazol in aqueous medium and due to first pass hepatic metabolism.

Usually danazol is administered orally and is therefore absorbed from the gastrointestinal tract. It has been observed that the absorption is influenced by the simultaneous ingestion of food. Thus, the extent of danazol absorption (AUC) was greatest when it was taken orally together with a meal.

In general, it is known that the absorption and bioavailability of a therapeutically active substance can be affected by a variety of factors when administered orally. Such factors include the presence of food in the gastrointestinal tract and, in general, the gastric residence time of a drug substance is significantly longer in the presence of food than in the fasted state. If the bioavailability of a drug substance is affected beyond a certain point due to the presence of food in the gastrointestinal tract, the drug substance is said to exhibit a food effect. Food effects are important because there is a risk associated with administering the drug substance to a patient who has eaten recently. The risk derives from the potential that absorption into the bloodstream may be adversely affected to the point that the patient risks insufficient absorption to remedy the condition for which the drug was administered.

Danazol is an inhibitor of both cytochrome P450 IIIA4 (CYP3A4) isoenzyme and P-glycoprotein. Many drug substances are substrates for P450 IIIA4 (CYP3A4) isoenzyme and P-glycoprotein and are extensively metabolized by the CYP3A4 isoenzyme in the gut wall and liver. Therefore, absorption and the subsequent elimination of systemically absorbed drug substances that are such substrates (e.g. sirolimus, tacrolimus etc.) may be influenced by other drug substances that affect this isoenzyme. Inhibitors of CYP3A4 may decrease the metabolism of e.g. sirolimus or tacrolimus and increase the drug levels, while inducers of CYP3A4 may increase the metabolism of e.g. sirolimus and decrease drug levels. Accordingly, drug substances like e.g. sirolimus may be administered together with one or more CYP3A4 inhibitors including e.g. danazol in order to improve the overall bioavailability.

For oral administration, danazol is currently formulated and marketed as capsules containing 100 mg or 200 mg under the trademark Danocrine®.

There remains a need for new pharmaceutical compositions comprising danazol and releasing danazol in a controlled manner so as to prolong the therapeutic effect after a single dose. Furthermore, there is a need for a new danazol compositions exhibiting increased bioavailability of the active compound and/or reduced or eliminated food effect. In particular it is desired to obtain and larger uptake of the active compound, and thereby provide for a reduction of the administered dose and/or dosages. Especially, since the daily dose of danazol is up to about 800 mg, a once daily tablet or capsule must have a size that normally is considered as possible, but inconvenient for a patient to swallow. Thus, a significant increase in bioavailability of danazol (as with the present technology) will lead to a reduction in the daily dose necessary to obtain the desired therapeutic effect and, according, lead to a reduction in the size of the tablet or capsule, which in turn lead to a better patient compliance Since danazol has been shown to exhibit a number of adverse side effects the latter is another important object. Further, pharmaceutical compositions comprising danazol and exhibiting a higher bioavailability of this compound may allow a reduction in the dose or dosage units taken by a patient, e.g. down to a single dose daily, and may also reduce or negate the need for food to be takes simultaneously with the dosage form thereby allowing patients more freedom on when the drug is taken. Furthermore, it is contemplated that fluctuations in the plasma concentration versus time profile may be significantly reduced due to a marked reduction in peak plasma concentration while the plasma concentration is maintained at a therapeutic level for an extended period of time.

Delaying the release of danazol to the distal part of duodenum may reduce the drug related gastrointestinal related side effects. Owing to the compositions and/or technology, this can be done without loosing systemic bioavailability.

### DESCRIPTION OF THE INVENTION

As mentioned above, there is a need for developing pharmaceutical danazol-containing compositions notably for oral use that lead to an improved treatment of conditions with danazol. An improved bioavailability will lead to an improved treatment because it will be possible to obtain the same therapeutic response with a decreased danazol dose and this in turn will lead to a significant reduction in dose-related side effects. Furthermore, only through a significant improvement in bioavailability it will be possible to aim at controlled release dosage forms for once daily administration. A therapeutic improvement would be to develop modified or delayed release compositions containing danazol, but in practice this is mostly of interest if it is possible overall to improve the bioavailability as discussed above.

The present invention provides pharmaceutical compositions and solid dosage forms for improved treatment of conditions that respond to danazol therapy. As danazol belongs to the therapeutic group of antigonadotropines, it is comtemplated that such compounds in general are suitable for incorporation into a pharmaceutical composition that has been designed to increase the bioavailability of the active substance. Thus, in one embodiment, the invention relates to pharmaceutical compositions comprising one or more antigondatotropines.

In the present context, the term “danazol” encompasses any relevant derivative or analogue of danazol. A composition of the invention may also include one or more further therapeutically, prophylactically and/or diagnostically active substances.

Known indications of danazol are e.g. endometriosis, hereditary angioedema, fibrocysic breast disease, premenstrual syndrome, breast cancer, thrombocytopenic purpura, thrombotic thrombocytopenic purpura. Danazol has also been indicated for use in acne, adenomyosis, alpha-1-antitrypsin deficiency, antiphospholipid antibody syndrome, antithrombin III deficiency, autoimmune acquired angioedema, breast gigantism, cholinergic urticaria, post-coital contraception, discoid lupus, Evan's syndrome, factor X deficiency, headache, migraine, hemolytic anemia, hemophilia, Henoch-Schonlein purpura, hypoprothrombinemia, infertility, mastalgia, menorrhagia, myelodysplasia, myeloid metaplasia, ovarian tumors, paroxysmal cold hemoglobinuria, prostate cancer, protein-C deficiency, pruritus, sideroblastic anemia, systemic lupus erythematosus, hemorrhagic telangiectasia, thrombocytopenia, idiopathic thrombocytopenic purpura, livedoid vasculitis, and vasomotor symptoms.

In one aspect, the present invention relates to a pharmaceutical composition in particulate form comprising danazol together with one or more pharmaceutically acceptable excipient, wherein the composition upon oral administration to a mammal in need thereof exhibits an AUC/AUC_{Control} value of at least about 1.5, the AUC values being determined under similar conditions. The composition used as a control is given in the same dosage and is a commercially available danazol composition intended for oral administration. In the present context, the control composition is Danocrine® capsules.

As it appears from the Examples herein the bioavailability obtained after administration of a composition according to the invention is markedly improved. Thus, in specific embodiments, the AUC/AUC_{Control} value is at least about 1.75 such as about 1.8 or more, about 1.9 or more, about 2.0 or more, about 2.5 or more, about 2.75 or more, about 3.0 or more, about 3.25 or more, about 3.5 or more, about 3.75 or more, about 4.0 or more, about 4.25 or more, about 4.5 or more, about 4.75 or more, about 5 or more, about 6 or more, about 7 or more, about 8 or more, about 9 or more, or about 10 or more, the AUC values being determined under similar conditions.

After oral administration of a pharmaceutical composition according to the present invention it is contemplated that the plasma concentration versus time profile show an extended period of time in which the plasma concentration is maintained within the therapeutic window (i.e. the plasma concentration leads to a therapeutic effect) without leading to serious unwanted side effects. Thus, a reduction in peak concentration may also be observed. In a specific embodiment, it may be of interest to provide a pharmaceutical composition (in particulate or e.g. tablet form) comprising danazol or a derivative or analogue thereof together with one or more pharmaceutically acceptable excipient, wherein the composition upon oral administration to a mammal in need thereof release danazol or a derivative or analogue thereof in a controlled manner and exhibits a Cₘₐₓ that is at the most about 80% of that of Cₘₐₓ for Danocrine® tablets such as, e.g., at the most about 75%, at the most about 70%, at the most about 65%, at the most about 60%, at the most about 55%, at the most about 50%, at the most about 45% or at the most about 40%.

However, a reduction in peak concentration should not lead to a decrease in therapeutic effect. Accordingly, the present invention also relates to a pharmaceutical composition, wherein W₅₀ is at least about 2 hours, such as, e.g., at least about 3 hours, at least about 4 hours, at least about 5 hours, at least about 6 hours, at least about 7 hours, at least about 8 hours, at least about 9 hours, at least about 10 hours, at least about 12 hours, at least about 14 hours, at least about 16 hours, at least about 18 hours or at least about 20 hours. Furthermore or moreover, a composition according to the invention has a C_{diff}=[Cₘₐₓ−Cₜ (t is at least 6 hours and at the most 9 hours, normally t is set to 7 hours)] that is less than that of Danocrine® tablets under the same conditions. If C_{diff} for Danocrine® tablets is set to 100 then C_{diff} of a composition according to the invention may be 90 or less such as, e.g., about 85 or less, about 80 or less, about 75 or less, about 70 or less, about 65 or less, about 60 or less, about 55 or less, about 50 or less, about 45 or less or about 40 or less.

Thus, it has surprisingly been found that the pharmaceutical compositions according to the invention exhibit surprisingly higher bioavailability compared to commercially available formulations such as Danocrine®. In fact the bioavailability of danazol can according to the invention be increased by over 500% compared with the said commercially available products. Accordingly, the current daily dose of danazol may be significantly reduced by administration of a composition of the invention. It is contemplated that the current daily dose of about 800 mg can be reduced to about 125-200 mg and due to this reduced amount it is possible to provide a composition for administration once with only one tablet or capsule.

A pharmaceutical composition according to the invention releases danazol in a controlled manner in order to extend the therapeutic action of danazol. In a particularly interesting aspect, the composition is in the form of a tablet. The release may suitably be pH independent, e.g. by providing the composition with a controlled release coating such as, e.g. a cellulose based coating like e.g. ethylcellulose, or by use of a controlled release matrix. A combination may of course also be employed.

In general, the change in bioavailability and/or the changes in other bioavailability related parameters are normally determined by in vivo studies in a suitable animal model testing the compositions in question together with e.g. Danocrine® or a similar commercially available danazol-containing product. The use of a dog model for establishing evidence of the bioavailability of certain formulations is general practice in the pharmaceutical industry.

The studies relevant for danazol are non-randomized, cross-over studies, where each dog is its own control. Four dogs and four treatments are normally applied. As no i.v. injections are given, the bioavailabilities obtained are relative.

Further it has surprisingly been found that the need for simultaneous food intake in order to secure a sufficient uptake of danazol is significantly reduced or even completely abolished.

Thus, the pharmaceutical compositions according to the invention provide significant higher bioavailability of danazol, which may reduce the daily intake of danazol, and reduce or abolish the need for administration in connection with food intake, which provide for a higher degree of freedom for the recipient of the pharmaceutical compositions, and consequently the patients acceptance and/or compliance may be significantly improved. Furthermore, the compositions provide a significant reduction in side effects, especially side effect related to a high peak concentration (such as, e.g., vomiting and nausea) and provide for an extended release of danazol leading to a better therapy such as, e.g., administration only once daily.

As mentioned above, besides improving the overall bioavailabily, one of the major challenges with respect to formulation of danazol compositions is to avoid an adverse food effect. In general, danazol is much better absorbed when it is administered orally together with food. A great variation in bioavailability is therefore seen following administration with or without food. This dependency makes it difficult to give precise guidelines as to how large a dose that should be administered and, furthermore, it requires information to the patient about the dosing regime. The present invention aims at providing compositions wherein the adverse food effect is reduced. Thus, the present invention provides a composition, which does not exhibit a significant adverse food effect after administration of the composition to a mammal in need of such a treatment as evidenced by a value of (AUC_{fed}/AUC_{fasted}) of at least about 0.85 with a lower 90% confidence limit of at least 0.75.

More specifically, a pharmaceutical composition according to the invention has a value of (AUC_{fed}/AUC_{fasted}) of about 0.9 or more such as, e.g., about 0.95 or more, about 0.97 or more or about 1 or more such as, e.g., up to about 1.1 or up to about 1.2.

A further advantage of a composition of the present invention is the possibility of obtaining an effective therapeutic response with a decreased dosage and/or a decreased administration frequency compared to traditional oral treatment. Accordingly, upon oral administration to a mammal in need thereof a pharmaceutical composition according to the invention releases danazol or an analogue thereof in a controlled manner and the composition is essentially bioequivalent with Danocrine® or a similar commercially available danazol-containing product when administered in a dose that is at the about most about 85% w/w such as, e.g., at the most about 80% w/w, at the most about 75%, at the most about 70% w/w, at the most about 65% w/w, at the most about 60% w/w, at the most about 55% w/w or at the most about 50% w/w of the dose of danazol administered in the form of Danocrine® or a similar commercially available danazol-containing product.

Parameters often used in bioequivalence studies are tₘₐₓ, cₘₐₓ, AUC_{0-infinity}, AUC₀₋ₜ. Other relevant parameters may be W₅₀, W₇₅ and/or MRT. Accordingly, at least one of these parameters may be applied when determining whether bioequivalence is present. Furthermore, in the present context, two compositions are regarded as bioequivalent if value of the parameter used is within 80-125% of that of Danocrine® or a similar commercially available danazol-containing product used in the test.

In the present context “tₘₐₓ” denotes the time to reach the maximal plasma concentration (cₘₐₓ) after administration; AUC_{0-infinity} denotes the area under the plasma concentration versus time curve from time 0 to infinity; AUC₀₋ₜ denotes the area under the plasma concentration versus time curve from time 0 to time t; W₅₀ denotes the time where the plasma concentration is 50% or more of Cₘₐₓ; W₇₅ denotes the time where the plasma concentration is 75% or more of Cₘₐₓ; and MRT denotes mean residence time for danazol (and/or a derivative and/or an analogue thereof).

In some embodiments of the invention, the compositions are designed to release danazol in a pH-dependent manner so as to avoid release in the stomach and delay the release until the composition after oral administration passes the stomach and reaches the small intestine. Delayed release is mainly brought about by some kind of enteric coating. Whereas semipermeable coating will show some kind of delayed release, it does not significantly delay release. Additionally it requires a certain amount of time to release the content. The coating sought for this invention, may be a pH dependant coating. This type of coating is very resistant to release of drug until a certain pH is reached. Within very few 1/10′th of pH, the film alters properties and becomes permeable. Examples of pH-sensitive polymers, which are relatively insoluble and impermeable at the pH of the stomach, but which are more soluble and permeable at the pH of the small intestine and colon include, but not limited to:

polyacrylamides, phthalate derivatives such as acid phthalates of carbohydrates, amylose acetate phthalate, cellulose acetate phthalate, other cellulose ester phthalates, cellulose ether phthalates, hydroxypropylcellulose phthalate, hydroxypropylethylcellulose phthalate, hydroxypropylmethylcellulose phthalate, methylcellulose phthalate, polyvinyl acetate phthalate, polyvinyl acetate hydrogen phthalate, sodium cellulose acetate phthalate, starch acid phthalate, styrene-maleic acid dibutyl phthalate copolymer, styrene-maleic acid polyvinylacetate phthalate copolymer, styrene and maleic acid copolymers, polyacrylic acid derivatives such as acrylic acid and acrylic ester copolymers, polymethacrylic acid and esters thereof, poly acrylic methacrylic acid copolymers, shellac, and vinyl acetate and crotonic acid copolymers.

pH-sensitive polymers of specific interest include shellac; phthalate derivatives, particularly cellulose acetate phthalate, polyvinylacetate phthalate, and hydroxypropylmethylcellulose phthalate; polyacrylic acid derivatives, particularly polymethyl methacrylate blended with acrylic acid and acrylic ester copolymers; and vinyl acetate and crotonic acid copolymers.

Increasing the bioavailability, the Area Under the Curve (AUC), will normally reduce the intra- and inter-variability related to absorption of a drug substance. This is particularly true; whenever the low and impaired bioavailability is a consequence of poor water solubility. It is contemplated that compositions according to the invention will provide CVs on Area under Curve data that are significantly smaller than with Danocrine® and like products.

Furthermore, it is envisaged that a pharmaceutical composition comprising danazol together with one or more pharmaceutically acceptable excipient—and wherein the composition upon oral administration to a mammal in need thereof releases danazol or an analogue thereof in a controlled manner (dependent on the design of the composition, this may be a pH-dependant or a pH-independent manner)-reduces inter- and/or intra-individual variations compared to those of Danocrine® administered under the same conditions and in a dose that provides an equivalent therapeutic effect.

In a specific aspect, the invention provides a pharmaceutical composition or a solid dosage form that releases danazol and/or a derivative or analogue thereof in an extended manner so as to enable a long maintenance of the therapeutic effect. Accordingly, the invention relates to a pharmaceutical composition (e.g. in particulate or in a solid dosage unit form like e.g. tablets or capsulers) form comprising danazol and/or an analogue thereof together with one or more pharmaceutically acceptable excipient, wherein the composition upon oral administration to a mammal in need thereof in a controlled manner releases at least about 50% w/w of the total amount of danazol and/or an analogue thereof within about 15 hours such as, e.g., within about 12 hours.

In specific embodiments of the invention, a composition releases danazol according to in one or more of the following requirements. The release may be in vivo in the gastrointestinal tract and/or in vitro as tested by a suitable in vitro dissolution test e.g. according to Ph.Eur.

i) At least about 50% w/w danazol is released after at least about 2 hours, such as, e.g., at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, or at least about 17 hours, and/or

ii) at least about 60% w/w danazol is released after at least about 2 hours, such as, e.g., at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, or at least about 17 hours, and/or

iii) at least about 70% w/w danazol is released after at least about 3 hours, such as, e.g., at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, or at least about 18 hours, and/or

iv) at least 80% w/w danazol is released after at least 4 hours, such as, e.g., at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours or at least 20 hours and/or

v) at least 85% w/w such as, e.g., at least about 90% w/w or at least about 95% w/w danazol is released after at least 5 hours, such as, e.g., at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, or at least about 22 hours, and/or

vi) at the most about 20% w/w is released within about 10 hours such as, e.g., within about 9 hours, within about 8 hours, within about 7 hours, within about 6 hours, within about 5 hours, within about 4 hours, within about 3 hours or within about 2 hours, and/or

vii) at the most about 30% w/w is released within about 12 hours such as, e.g., within about 11 hours, within about 10 hours, within about 9 hours, within about 8 hours, within about 7 hours, within about 6 hours, within about 5 hours, within about 4 hours, or within about 3 hours, and/or

viii) at the most about 40% w/w is released within about 13 hours such as, e.g., within about 12 hours, within about 11 hours, within about 10 hours, within about 9 hours, within about 8 hours, within about 7 hours, within about 6 hours, within about 5 hours, or within about 4 hours, and/or

ix) at the most about 50% w/w is released within about 14 hours such as, e.g., within about 13 hours, within about 12 hours, within about 11 hours, within about 10 hours, within about 9 hours, within about 8 hours, within about 7 hours, within about 6 hours, within about hours, or within about 4 hours, and/or

x) at the most 15% w/w is released within the first hour after administration or after start of the in vitro dissolution test, and/or

xi) at the most 20% w/w is released within 2 hours after administration or after start of the in vitro dissolution test, and/or

xii) at the most 25% w/w such as, e.g., from about 5% to about 25% w/w is released within 3 hours after administration or after start of the in vitro dissolution test, and/or

xiii) at the most 30% w/w such as, e.g., from about 10% to about 25% w/w is released within 4 hours after administration or after start of the in vitro dissolution test, and/or

xiv) at the most 45% w/w such as, e.g., from about 20% to about 45% w/w is released within 6 hours after administration or after start of the in vitro dissolution test, and/or

xv) at the most 55% w/w such as, e.g., from about 35% to about 55% w/w is released within 8 hours after administration or after start of the in vitro dissolution test, and/or

xvi) at the most 70% w/w such as, e.g., from about 35% to about 70% w/w is released within 10 hours after administration or after start of the in vitro dissolution test, and/or

xvii) at the most 80% w/w such as, e.g., from about 40% to about 80% w/w is released within 12 hours after administration or after start of the in vitro dissolution test.

In other specific embodiments, a composition according to the invention—upon oral administration to a mammal in need thereof—releases at least about 50% w/w of the total amount of danazol and/or an analogue thereof within about 10 hours such as, e.g., within about 8 hours, within about 6 hours, within about 4 hours or within about 3 hours.

In a further embodiment, upon oral administration to a mammal in need thereof a pharmaceutical composition according to the invention releases at least about 55% w/w such as, e.g., about 60% w/w or more, about 65% w/w or more, about 70% w/w or more, about 75% w/w or more or about 80% w/w or more of the total amount of danazol and/or an analogue thereof within about 15 hours such as, e.g., within about 12 hours, within about 10 hours, within 8 hours or within about 6 hours.

Furthermore or alternatively, at least about 50% w/w of the total amount of danazol and/or an analogue thereof is released within 15 hours such as, e.g., within about 12 hours, when tested in an in vitro dissolution test and employing a dissolution medium comprising a buffer having pH 7.5. Guidance for a suitable dissolution test is described in the Examples herein, but variations with respect to the specific method employed and the ingredients contained in the dissolution medium etc. are within the scope of the present invention. A person skilled in the art will know how to carry out a suitable dissolution test e.g. with guidance from USP, Ph.Eur. and the like. Suitable conditions for the in vitro dissolution test are employing USP dissolution test (paddle method) and a buffer pH 7.5 containing 0.75% sodium lauryl sulfate as dissolution medium.

In other embodiments, the following conditions are fulfilled with respect to in vitro dissolution test:

i) at least about 50% w/w of the total amount of danazol or an analogue thereof is released within about 10 hours such as, e.g., within about 8 hours, within about 6 hours, within about 4 hours, within about 3 hours or within about 2 hours, when tested in an in vitro dissolution test and employing a dissolution medium comprising a buffer having pH 7.5

ii) at least about 50% w/w of the total amount of danazol or an analogue thereof is released within about 1.5 hours such as, e.g., within about 1 hour, within about 0.75 hours, within about 0.5 hours or within about 20 minutes, when tested in an in vitro dissolution test and employing a dissolution medium comprising a buffer having pH 7.5.

iii) at least about 55% w/w such as, e.g., about 60% w/w or more, about 65% w/w or more, about 70% w/w or more, about 75% w/w or more or about 80% w/w or more of the total amount of danazol or an analogue thereof is released within about 15 hours such as, e.g., within about 12 hours, within about 10 hours, within 8 hours or within about 6 hours, when tested in an in vitro dissolution test and employing a dissolution medium comprising a buffer having pH 7.5

iv) at least about 55% w/w such as, e.g., about 60% w/w or more, about 65% w/w or more, about 70% w/w or more, about 75% w/w or more or about 80% w/w or more of the total amount of danazol or an analogue thereof is released within about 5 hours such as, e.g., within about 4 hours, within about 3 hours, within about 2 hours, within about 1 hours or within about 30 minutes, when tested in an in vitro dissolution test and employing a dissolution medium comprising a buffer having pH 7.5, and/or

v) at least about 20% w/w such as, e.g., at least about 25% w/w, at least about 30% w/w, at least about 35% w/w or at least about 40% w/w of the total amount of danazol or an analogue thereof is released within the first 3 hours such as, e.g., within the first 2 hours or within the first hour when tested in an in vitro dissolution test and employing a dissolution medium comprising a buffer having pH 7.5.

In an interesting embodiment, the composition is designed to have a delayed release of danazol and/or an analogue thereof. Therefore, the invention also includes a pharmaceutical composition comprising danazol and/or an analogue thereof together with one or more pharmaceutically acceptable excipient, wherein the composition upon oral administration to a mammal in need thereof has a prolonged/delayed release of danazol and/or an analogue thereof so that at the most 10% w/w such as, e.g., at the most about 7.5% w/w or at the most about 5% w/w of the total amount of danazol or an analogue thereof is released within the first two hours such as, e.g., within the first hour after administration.

In other embodiments, the following conditions are fulfilled with respect to in vitro dissolution test performed:

i) at the most about 30% w/w such as, e.g., at the most about 25% w/w, at the most about 20% w/w, at the most about 15% w/w or at the most about 10% w/w of danazol or an analogue thereof is released within 2 hours in an in vitro dissolution test employing a dissolution medium having a pH of at the most about 5 such as, e.g. at the most about 4.5, at the most about 4, at the most about 3.5, at the most about 3, at the most about 2 or at the most about 1.5,

ii) at the most about 10% w/w such as, e.g., at the most about 7.5% w/w, at the most about 5% w/w or at the most about 2.5% w/w of danazol or an analogue thereof is released within 2 hours in an in vitro dissolution test employing a dissolution medium having a pH of at the most about 5 such as, e.g. at the most about 4.5, at the most about 4, at the most about 3.5, at the most about 3, at the most about 2 or at the most about 1.5

iii) at the most about 60% w/w such as, e.g., at the most about 50% w/w, at the most about 40% w/w or at the most about 30% w/w of danazol or an analogue thereof is released within 15 hours such as, e.g., within about 12 hours, when tested in an in vitro dissolution test employing a dissolution medium having a pH of at the most about 4.5 such as, e.g. at the most about 4.0, at the most about 3.5, at the most about 3, at the most about 2 or at the most about 1.5

iv) at the most about 40% w/w such as, e.g., at the most about 30% w/w, at the most about 25% w/w or at the most about 20% w/w of danazol or an analogue thereof is released within 6 hours when tested in an in vitro dissolution test employing a dissolution medium having a pH of at the most about 4.5 such as, e.g. at the most about 4.0, at the most about 3.5, at the most about 3, at the most about 2 or at the most about 1.5, and/or

v) at the most about 30% w/w such as, e.g., at the most about 25% w/w, at the most about 20% w/w or at the most about 15% w/w of danazol or an analogue thereof is released within 4 hours when tested in an in vitro dissolution test employing a dissolution medium having a pH of at the most about 4.5 such as, e.g. at the most about 4.0, at the most about 3.5, at the most about 3, at the most about 2 or at the most about 1.5.

The pharmaceutical compositions may be prepared by any convenient method such as, e.g. granulation, mixing, spray drying etc. A particularly useful method is the method described in WO 03/004001. Herein is described a process for the preparation of particulate material by a controlled agglomeration method, i.e. a method, which enables a controlled growth in particle size. The method involves spraying a first composition comprising e.g. danazol and a carrier, which has been melted, onto a second solid carrier medium. Normally, the meltable carrier has a melting point of at least 5° C. but lower than the melting point of danazol. The melting point of the carrier may be in the range of 10° C. to 150° C., such as, e.g., in the range of 30° C. to 100° C. or in the range of 40° C. to 50° C. is most preferred.

It is within the skills of the average practioner to select a suitable carrier being pharmaceutical acceptable, capable of dispersing or at least partly dissolving danazol and having a melting point in the desired range using general knowledge and routine experimentation. Suitable candidate for carriers are described in WO 03/004001, which is herein incorporated by reference.

In the present context, suitable carriers are e.g. those mentioned as an oily material (as discussed later herein) as well as those disclosed in WO 03/004001.

An advantage of using the controlled agglomeration method described in WO 03/004001 is that it is possible to apply a relatively large amount of a melt to a particulate material without having an undesirable growth in particle size. Accordingly, in one embodiment of the invention, the particulate material of a pharmaceutical composition has a geometric weight mean diameter d_{gw} of ≧10 μm such as, e.g. ≧20 μm, from about 20 to about 2000, from about 30 to about 2000, from about 50 to about 2000, from about 60 to about 2000, from about 75 to about 2000 such as, e.g. from about 100 to about 1500 μm, from about 100 to about 1000 μm or from about 100 to about 700 μm, or at the most about 400 μm or at the most 300 μm such as, e.g., from about 50 to about 400 μm such as, e.g., from about 50 to about 350 μm, from about 50 to about 300 μm, from about 50 to about 250 μm or from about 100 to about 300 μm.

Pharmaceutically Acceptable Excipients

In the present context the terms “pharmaceutically acceptable excipient” are intended to denote any material, which is inert in the sense that it substantially does not have any therapeutic and/or prophylactic effect per se. Such an excipient may be added with the purpose of making it possible to obtain a pharmaceutical, cosmetic and/or foodstuff composition, which have acceptable technical properties.

Examples of suitable excipients for use in a composition or solid dosage form according to the invention include fillers, diluents, disintegrants, binders, lubricants etc. or mixture thereof. As the composition or solid dosage form according to the invention may be used for different purposes, the choice of excipients is normally made taken such different uses into considerations. Other pharmaceutically acceptable excipients for suitable use are e.g. acidifying agents, alkalizing agents, preservatives, antioxidants, buffering agents, chelating agents, coloring agents, complexing agents, emulsifying and/or solubilizing agents, flavors and perfumes, humectants, sweetening agents, wetting agents etc.

Examples of suitable fillers, diluents and/or binders include lactose (e.g. spray-dried lactose, α-lactose, β-lactose, Tabletose®, various grades of Pharmatose®, Microtose® or Fast-Floc®), microcrystalline cellulose (various grades of Avicel®, Elcema®, Vivacel®, Ming Tai® or Solka-Floc®), hydroxypropylcellulose, L-hydroxypropylcellulose (low substituted), hydroxypropyl methylcellulose (HPMC) (e.g. Methocel E, F and K, Metolose SH of Shin-Etsu, Ltd, such as, e.g. the 4,000 cps grades of Methocel E and Metolose 60 SH, the 4,000 cps grades of Methocel F and Metolose 65 SH, the 4,000, 15,000 and 100,000 cps grades of Methocel K; and the 4,000, 15,000, 39,000 and 100,000 grades of Metolose 90 SH), methylcellulose polymers (such as, e.g., Methocel A, Methocel A4C, Methocel A15C, Methocel A4M), hydroxyethylcellulose, sodium carboxymethylcellulose, carboxymethylene, carboxymethylhydroxyethylcellulose and other cellulose derivatives, sucrose, agarose, sorbitol, mannitol, dextrins, maltodextrins, starches or modified starches (including potato starch, maize starch and rice starch), calcium phosphate (e.g. basic calcium phosphate, calcium hydrogen phosphate, dicalcium phosphate hydrate), calcium sulfate, calcium carbonate, sodium alginate, collagen etc.

Specific examples of diluents are e.g. calcium carbonate, dibasic calcium phosphate, tribasic calcium phosphate, calcium sulfate, microcrystalline cellulose, powdered cellulose, dextrans, dextrin, dextrose, fructose, kaolin, lactose, mannitol, sorbitol, starch, pregelatinized starch, sucrose, sugar etc.

Specific examples of disintegrants are e.g. alginic acid or alginates, microcrystalline cellulose, hydroxypropyl cellulose and other cellulose derivatives, croscarmellose sodium, crospovidone, polacrillin potassium, sodium starch glycolate, starch, pregelatinized starch, carboxymethyl starch (e.g. Primogel® and Explotab®) etc.

Specific examples of binders are e.g. acacia, alginic acid, agar, calcium carrageenan, sodium carboxymethylcellulose, microcrystalline cellulose, dextrin, ethylcellulose, gelatin, liquid glucose, guar gum, hydroxypropyl methylcellulose, methylcellulose, pectin, PEG, povidone, pregelatinized starch etc.

Glidants and lubricants may also be included in the second composition. Examples include stearic acid, magnesium stearate, calcium stearate or other metallic stearate, talc, waxes and glycerides, light mineral oil, PEG, glyceryl behenate, colloidal silica, hydrogenated vegetable oils, corn starch, sodium stearyl fumarate, polyethylene glycols, alkyl sulfates, sodium benzoate, sodium acetate etc.

Other excipients which may be included in a composition or solid dosage form of the invention are e.g. flavoring agents, coloring agents, taste-masking agents, pH-adjusting agents, buffering agents, preservatives, stabilizing agents, anti-oxidants, wetting agents, humidity-adjusting agents, surface-active agents, suspending agents, absorption enhancing agents, agents for modified release etc.

Other additives in a composition or a solid dosage form according to the invention may be antioxidants like e.g. ascorbic acid, ascorbyl palmitate, butylated hydroxyanisole, butylated hydroxytoluene, hypophosphorous acid, monothioglycerol, potassium metabisulfite, propyl gallate, sodium formaldehylde sulfoxylate, sodium metabisulfite, sodium thiosulfate, sulfur dioxide, tocopherol, tocopherol acetate, tocopherol hemisuccinate, TPGS or other tocopherol derivatives, etc. The carrier composition may also contain e.g. stabilising agents. The concentration of an antioxidant and/or a stabilizing agent in the carrier composition is normally from about 0.1% w/w to about 5% w/w.

A composition or solid dosage form according to the invention may also include one or more surfactants or substances having surface-active properties. It is contemplated that such substances are involved in the wetting of the slightly soluble active substance and thus, contributes to improved solubility characteristics of the active substance.

Examples of surfactants are mentioned below.

Suitable excipients for use in a composition or a solid dosage form according to the invention are surfactants such as, e.g., hydrophobic and/or hydrophilic surfactants as those disclosed in WO 00/50007 in the name of Lipocine, Inc. Examples on suitable surfactants are
i) polyethoxylated fatty acids such as, e.g. fatty acid mono- or diesters of polyethylene glycol or mixtures thereof such as, e.g. mono- or diesters of polyethylene glycol with lauric acid, oleic acid, stearic acid, myristic acid, ricinoleic acid, and the polyethylene glycol may be selected from PEG 4, PEG 5, PEG 6, PEG 7, PEG 8, PEG 9, PEG 10, PEG 12, PEG 15, PEG 20, PEG 25, PEG 30, PEG 32, PEG 40, PEG 45, PEG 50, PEG 55, PEG 100, PEG 200, PEG 400, PEG 600, PEG 800, PEG 1000, PEG 2000, PEG 3000, PEG 4000, PEG 5000, PEG 6000, PEG 7000, PEG 8000, PEG 9000, PEG 1000, PEG 10,000, PEG 15,000, PEG 20,000, PEG 35,000,
ii) polyethylene glycol glycerol fatty acid esters, i.e. esters like the above-mentioned but in the form of glyceryl esters of the individual fatty acids;
iii) glycerol, propylene glycol, ethylene glycol, PEG or sorbitol esters with e.g. vegetable oils like e.g. hydrogenated castor oil, almond oil, palm kernel oil, castor oil, apricot kernel oil, olive oil, peanut oil, hydrogenated palm kernel oil and the like,
iv) polyglycerized fatty acids like e.g. polyglycerol stearate, polyglycerol oleate, polyglycerol ricinoleate, polyglycerol linoleate,
v) propylene glycol fatty acid esters such as, e.g. propylene glycol monolaurate, propylene glycol ricinoleate and the like,
vi) mono- and diglycerides like e.g. glyceryl monooleate, glyceryl dioleae, glyceryl mono- and/or dioleate, glyceryl caprylate, glyceryl caprate etc.; vii) sterol and sterol derivatives;
viii) polyethylene glycol sorbitan fatty acid esters (PEG-sorbitan fatty acid esters) such as esters of PEG with the various molecular weights indicated above, and the various Tween® series;
ix) polyethylene glycol alkyl ethers such as, e.g. PEG oleyl ether and PEG lauryl ether;
x) sugar esters like e.g. sucrose monopalmitate and sucrose monolaurate;
xi) polyethylene glycol alkyl phenols like e.g. the Triton® X or N series;
xii) polyoxyethylene-polyoxypropylene block copolymers such as, e.g., the Pluronic® series, the Synperonic® series, Emkalyx®, Lutrol®, Supronic® etc. The generic term for these polymers is “poloxamers” and relevant examples in the present context are Poloxamer 105, 108, 122, 123, 124, 181, 182, 183, 184, 185, 188, 212, 215, 217, 231, 234, 235, 237, 238, 282, 284, 288, 331, 333, 334, 335, 338, 401, 402, 403 and 407;
xiii) sorbitan fatty acid esters like the Span® series or Ariacel® series such as, e.g. sorbinan monolaurate, sorbitan monopalmitate, sorbitan monooleate, sorbitan monostearate etc.;
xiv) lower alcohol fatty acid esters like e.g. oleate, isopropyl myristate, isopropyl palmitate etc.;
xv) ionic surfactants including cationic, anionic and zwitterionic surfactants such as, e.g. fatty acid salts, bile salts, phospholipids, phosphoric acid esters, carboxylates, sulfates and sulfonates etc.

When a surfactant or a mixture of surfactants is present in a composition or a solid dosage form of the invention, the concentration of the surfactant(s) is normally in a range of from about 0.1-80% w/w such as, e.g., from about 0.1 to about 20% w/w, from about 0.1 to about 15% w/w, from about 0.5 to about 10% w/w, or alternatively, from about 0.10 to about 80% w/w such as, e.g. from about 10 to about 70% w/w, from about 20 to about 60% W/w or from about 30 to about 50% w/w.

In a specific aspect of the invention, the at least one of the one or more pharmaceutically acceptable excipient is selected from the group consisting of silica acid or a derivative or salt thereof including silicates, silicon dioxide and polymers thereof; magnesium aluminosilicate and/or magnesium aluminometasilicate, bentonite, kaolin, magnesium trisilicate, montmorillonite and/or saponite.

Such materials are is especially useful as a sorption material for oily materials in pharmaceuticals, cosmetics and/or foodstuff. In a specific embodiment, the material is used as a sorption material for oily materials in pharmaceuticals. The material that has the ability to function as a sorption material for oily materials is also denoted “oil sorption material”. Furthermore, in the present context the term “sorption” is used to denote “absorption” as well as “adsorption”. It should be understood that whenever one of the terms is used it is intended to cover the phenomenon absorption as well as adsorption.

Notably, the pharmaceutically acceptable excipient may comprise a silica acid or a derivative or salt thereof such as, e.g., silicon dioxide or a polymer thereof as a pharmaceutically acceptable excipient. Dependent on the quality employed a silicon dioxide may be a lubricant or it may be an oil sorption material. Qualities fulfilling the latter function seem to be most important.

In a specific embodiment, a composition or solid dosage form according to invention comprises a pharmaceutically acceptable excipient that is a silicon dioxide product that has properties corresponding to Zeofree® 5161A, Zeofree® 5162, Zeofree® 5175A, Zeopharm® 80 (available from J. M. Huber, Hamina, Finland), Aeroperl® 300, Sident® 22S, Sipernat®1160, Sipernat® 160PQ, Sipernat® 22, Sipernat® 22 LS, Sipernat® 22, Sipernat® 22 LS, Sipernat® 22S, Sipernat® 2200, Sipernat® 310, Sipernat® 320, Sipernat® 320 DS, Sipernat® 325 C, Sipernat® 35, Sipernat® 350, Sipernat® 360, Sipernat® 383 D8, Sipernat® 44, Sipernat® 44MS, Sipernat® 50, Sipernat® 50S, Sipernat® 50 S, Sipernat® 500 LS, or Sipernat® 570 (available from Degussa, Frankfurt, Germany).

As it appears from the examples herein, a very suitable material is Aeroperl® 300 (including materials with properties like or corresponding to those of Aeroperl® 300).

Use of an oil sorption material in compositions or dosage forms according to the invention is very advantageous for the preparation of pharmaceutical, cosmetic, nutritional and/or food compositions, wherein the composition comprises oily material. One of the advantages is that is it possible to incorporate a relatively large amount of oily material and still have a material that is solid. Thus, it is possible to prepare solid compositions with a relatively high load of oily materials by use of an oil sorption material according to the invention. Within the pharmaceutical field it is an advantage to be able to incorporate a relatively large amount of an oily material in a solid composition especially in those situation where the active substance does not have suitable properties with respect to water solubility (e.g. poor water solubility), stability in aqueous medium (i.e. degradation occurs in aqueous medium), oral bioavailability (e.g. low bioavailability) etc., or in those situations where it is desired to modify the release of an active substance from a composition in order to obtain a controlled, delayed, sustained and/or pulsed delivery of the active substance. Thus, in a specific embodiment it is used in the preparation of pharmaceutical compositions.

The oil sorption material for use in the processing into solid compositions normally absorbs about 5% w/w or more, such as, e.g., about 10% w/w or more, about 15% w/w or more, about 20% w/w or more, about 25% w/w or more, about 30% w/w or more, about 35% w/w or more, about 40% w/w or more, about 45% w/w or more, about 50 w/w or more, about 55% w/w or more, about 60% w/w or more, about 65% w/w or more, about 70% w/w or more, about 75% w/w or more, about 80% w/w or more, about 85% w/w or more, about 90% w/w or more or about 95% w/w or more of an oil or an oily material and is still a solid material.

An important aspect of the invention is compositions or solid dosage forms comprising an oily material.

Oily Materials

In the present context the term “oily materials” is used in a very broad sense including oils, waxes, semi-solid materials and materials that normally are used as solvents (such as organic solvents) or cosolvents within the pharmaceutical industry, and the term also includes therapeutically and/or prophylactically active substances that are in liquid form at ambient temperature; furthermore the term includes emulsions like e.g. microemulsions and nanoemulsions and suspensions. The oily materials that can be absorbed are normally liquid at ambient or elevated temperature (for practical reasons the max. temperature is about 250° C.). They may be hydrophilic, lipophilic, hydrophobic and/or amphiphilic materials.

The oily material that are suitable for use in the present context are substances or materials, which have a melting point of at least about 0° C. and at the most about 250° C.

In specific embodiments of the invention, the oily material has a melting point of about 5° C. or more such as, e.g., about 10° C. or more, about 15° C. or more, about 20° C. or more or about 25° C. or more.

In further embodiments of the invention, the oily material has a melting point of at least about 25° C. such as, e.g., at least about 30° C. at least about 35° C. or at least about 40° C. For practical reasons, the melting point may normally not be too high, thus, the oily material normally has a melting point of at the most about 300° C. such as, e.g., at the most about 250° C., at the most about 200° C., at the most about 150° C. or at the most about 100° C. If the melting point is higher a relatively high temperature may promote e.g. oxidation or other kind of degradation of an active substance in those cases where e.g. a therapeutically and/or prophylactically active substance is included.

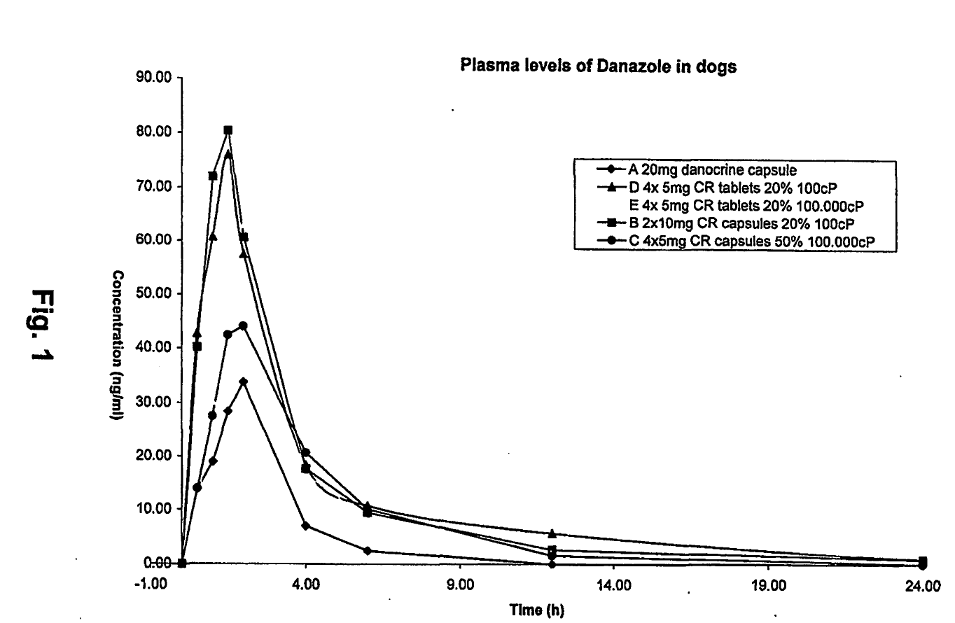

In the present context, the melting point is determined by DSC (Differential Scanning Calorimetry). The melting point is determined as the temperature at which the linear increase of the DSC curve intersects the temperature axis (see FIG. 1 for further details).

Interesting oily materials are generally substances, which are used in the manufacture of pharmaceuticals as so-called melt binders or solid solvents (in the form of solid dosage form), or as co-solvents or ingredients in pharmaceuticals for topical use.

It may be hydrophilic, hydrophobic and/or have surface-active properties. In general hydrophilic and/or hydrophobic oily materials are suitable for use in the manufacture of a pharmaceutical composition comprising a therapeutically and/or prophylactically active substance that has a relatively low aqueous solubility and/or when the release of the active substance from the pharmaceutical composition is designed to be immediate or non-modified. Hydrophobic oily materials, on the other hand, are normally used in the manufacture of a modified release pharmaceutical composition. The above-given considerations are simplified to illustrate general principles, but there are many cases where other combinations of oily materials and other purposes are relevant and, therefore, the examples above should not in any way limit the invention.

Typically, a suitable hydrophilic oily material is selected from the group consisting of: polyether glycols such as, e.g., polyethylene glycols, polypropylene glycols; polyoxyethylenes; polyoxypropylenes; poloxamers and mixtures thereof, or it may be selected from the group consisting of: xylitol, sorbitol, potassium sodium tartrate, sucrose tribehenate, glucose, rhamnose, lactitol, behenic acid, hydroquinon monomethyl ether, sodium acetate, ethyl fumarate, myristic acid, citric acid, Gelucire 50/13, other Gelucire types such as, e.g., Gelucire 44/14 etc., Gelucire 50/10, Gelucire 62/05, Sucro-ester 7, Sucro-ester 11, Sucro-ester 15, maltose, mannitol and mixtures thereof.

A suitable hydrophobic oily material may be selected from the group consisting of: straight chain saturated hydrocarbons, sorbitan esters, paraffins; fats and oils such as e.g., cacao butter, beef tallow, lard, polyether glycol esters; higher fatty acid such as, e.g. stearic acid, myristic acid, palmitic acid, higher alcohols such as, e.g., cetanol, stearyl alcohol, low melting point waxes such as, e.g., glyceryl monostearate, glyceryl monooleate, hydrogenated tallow, myristyl alcohol, stearyl alcohol, substituted and/or unsubstituted monoglycerides, substituted and/or unsubstituted diglycerides, substituted and/or unsubstituted triglycerides, yellow beeswax, white beeswax, carnauba wax, castor wax, japan wax, acetylate monoglycerides; NVP polymers, PVP polymers, acrylic polymers, or a mixture thereof.

In an interesting embodiment, the oily material is a polyethylene glycol having an average molecular weight in a range of from about 400 to about 35,000 such as, e.g., from about 800 to about 35,000, from about 1,000 to about 35,000 such as, e.g., polyethylene glycol 1,000, polyethylene glycol 2,000, polyethylene glycol 3,000, polyethylene glycol 4,000, polyethylene glycol 5,000, polyethylene glycol 6000, polyethylene glycol 7,000, polyethylene glycol 8,000, polyethylene glycol 9,000 polyethylene glycol 10,000, polyethylene glycol 15,000, polyethylene glycol 20,000, or polyethylene glycol 35,000. In certain situations polyethylene glycol may be employed with a molecular weight from about 35,000 to about 100,000.

In another interesting embodiment, the oily material is polyethylene oxide having a molecular weight of from about 2,000 to about 7,000,000 such as, e.g. from about 2,000 to about 100,000, from about 5,000 to about 75,000, from about 10,000 to about 60,000, from about 15,000 to about 50,000, from about 20,000 to about 40,000, from about 100,000 to about 7,000,000 such as, e.g., from about 100,000 to about 1,000,000, from about 100,000 to about 600,000, from about 100,000 to about 400,000 or from about 100,000 to about 300,000.

In another embodiment, the oily material is a poloxamer such as, e.g. Poloxamer 188, Poloxamer 237, Poloxamer 338 or Poloxamer 407 or other block copolymers of ethylene oxide and propylene oxide such as the Pluronic® and/or Tetronic® series. Suitable block copolymers of the Pluronic® series include polymers having a molecular weight of about 3,000 or more such as, e.g. from about 4,000 to about 20,000 and/or a viscosity (Brookfield) from about 200 to about 4,000 cps such as, e.g., from about 250 to about 3,000 cps. Suitable examples include Pluronic® F38, P65, P68LF, P75, F77, P84, P85, F87, F88, F98, P103, P104, P105, F108, P123, F123, F127, 10R8, 17R8, 25R5, 25R8 etc. Suitable block copolymers of the Tetronic® series include polymers having a molecular weight of about 8,000 or more such as, e.g., from about 9,000 to about 35,000 and/or a viscosity (Brookfield) of from about 500 to about 45,000 cps such as, e.g., from about 600 to about 40,000. The viscosities given above are determined at 60° C. for substances that are pastes at room temperature and at 77° C. for substances that are solids at room temperature.

The oily material may also be a sorbitan ester such as, e.g., sorbitan di-isostearate, sorbitan dioleate, sorbitan monolaurate, sorbitan monoisostearate, sorbitan monooleate, sorbitan monopalmitate, sorbitan monostearate, sorbitan sesqui-isostearate, sorbitan sesquioleate, sorbitan sesquistearate, sorbitan tri-isostearate, sorbitan trioleate, sorbitan tristearate or mixtures thereof.

The oily material may of course comprise a mixture of different oily materials such as, e.g., a mixture of hydrophilic and/or hydrophobic materials.

Other suitable oily materials may be solvents or semi-solid excipients like, e.g. propylene glycol, polyglycolised glycerides including Gelucire 44/14, complex fatty materials of plant origin including theobroma oil, carnauba wax, vegetable oils like e.g. almond oil, coconut oil, corn oil, cottonseed oil, sesame oil, soya oil, olive oil, castor oil, palm kernels oil, peanut oil, rape oil, grape seed oil etc., hydrogenated vegetable oils such as, e.g. hydrogenated peanut oil, hydrogenated palm kernels oil, hydrogenated cottonseed oil, hydrogenated soya oil, hydrogenated castor oil, hydrogenated coconut oil; natural fatty materials of animal origin including beeswax, lanolin, fatty alcohols including cetyl, stearyl, lauric, myristic, palmitic, stearic fatty alcohols; esters including glycerol stearate, glycol stearate, ethyl oleate, isopropyl myristate; liquid interesterified semi-synthetic glycerides including Miglycol 810/812; amide or fatty acid alcolamides including stearamide ethanol, diethanolamide of fatty coconut acids, acetic acid esters of mono and di-glycerides, citric acid esters of mono and di-glycerides, lactic acid esters of mono and diglycerides, mono and di-glycerides, poly-glycerol esters of fatty acids, poly-glycerol poly-ricinoleate, propylene glycol esters of fatty acids, sorbitan monostearates, sorbitan tristearates, sodium stearoyl lactylates, calcium stearoyl lactylates, diacetyl tartaric acid esters of mono and di-glycerides etc.

Normally, a pharmaceutical composition or a solid dosage form according to the invention has a concentration of the oily material in the composition of about 5% W/W or more such as, e.g., about 10% w/w or more, about 15% w/w or more, about 20% w/w or more, about 25% w/w or more, about 30% w/w or more, about 35% w/w or more, about 40% w/w or more, about 45% w/w or more, about 50 w/w or more, about 55% w/w or more, about 60% w/w or more, about 65% w/w or more, about 70% w/w or more, about 75% w/w or more, about 80% w/w or more, about 85% w/w or more, about 90% w/w or more or about 95% w/w or more.

In specific embodiments the concentration of the oily material in a composition or solid dosage form of the invention is in a range from about 20% to about 80% w/w such as, e.g., from about 25% to about 75% w/w.

One of the advantages is that is it possible to incorporate a relatively large amount of oily material and still have a material that is solid. Thus, it is possible to prepare solid compositions with a relatively high load of oily material by use of an oil sorption material according to the invention. Within the pharmaceutical field it is an advantage to be able to incorporate a relatively large amount of an oily material in a solid composition especially in those situation where the active substance does not have suitable properties with respect to water solubility (e.g. poor water solubility), stability in aqueous medium (i.e. degradation occurs in aqueous medium), oral bioavailability (e.g. low bioavailability) etc., or in those situations where it is desired to modify the release of an active substance from a composition in order to obtain a controlled, delayed, sustained and/or pulsed delivery of the active substance.

A further advantage is that the particulate material obtained is a free-flowing powder and therefore readily processable into e.g. solid dosage forms such as tablets, capsules or sachets. Normally, the particulate material has properties that are suitable in order to manufacture tablets by direct compression without addition of large amounts of further additives. A suitable test for test the flowability of the particulate material is the method described in Ph.Eur. and measuring the flow rate of the material out of a funnel with a nozzle (orifice) diameter of 10.0 mm.

In an important embodiment of the invention, at least a part of danazol and/or an analogue thereof is present in the composition in the form of a solid dispersion including a molecular dispersion and a solid solution. Normally, 10% or more such as, e.g., 20% or more, 30% or more, 40% or more, 50% or more, 60% or more, 70% or more, 80% or more, 90% or more such as, e.g., 95% or more or about 100% w/w of danazol and/or an analogue thereof is present in the composition in the form of a solid dispersion.

A solid dispersion may be obtained in different ways e.g. by employing organic solvents or by dispersing or dissolving the active substance in another suitable medium (e.g. an oily material in liquid form at room temperature or at elevated temperatures).

Description of a Solid Dispersion Based on Organic Solvents

Solid dispersions (solvent method) are prepared by dissolving a physical mixture of the active substance (e.g. a drug substance) and the carrier in a common organic solvent, followed by evaporation of the solvent. The carrier is often a hydrophilic polymer. Suitable organic solvents include pharmaceutical acceptable solvent in which the active substance is soluble such as methanol, ethanol, methylene chloride, chloroform, ethylacetate, acetone or mixtures thereof.

Suitable water soluble carriers include polymers such as polyethylene glycol, poloxamers, polyoxyethylene stearates, poly-8-caprolactone, polyvinylpyrrolidone (PVP), polyvinylpyrrolidone-polyvinylacetate copolymer PVP-PVA (Kollidon VA64), poly-methacrylic polymers (Eudragit RS, Eudragit RL, Eudragit NE, Eudragit E) and polyvinyl alcohol (PVA), hydroxypropyl cellulose (HPC), hydroxypropyl methyl cellulose (HPMC), methyl cellulose, and poly(ethylene oxide) (PEO).

Polymers containing acidic functional groups may be suitable for solid dispersions, which release the active substance in a preferred pH range providing acceptable absorption in the intestines. Such polymers may be one or more selected from the group comprising hydroxypropyl methylcellulose phtalate (HMPCP), polyvinyl acetate phtalate (PVAP), hydroxypropylmethylcellulose acetate succinate (HPMCAS), alginate, carbomer, carboxymethylcellulose, methacrylic acid copolymer (Eudragit L, Eudragit S), shellac, cellulose acetate phthalate (CAP), starch glycolate, polacrylin, methyl cellulose acetate phtalate, hydroxypropyulcellulose acetate phthalate, cellulose acetate terephtahalate, cellulose acetate isophthalate and cellulose acetate trimellitate.

In relations to amounts of the active substance and the polymer in the solid dispersion, the weight ratio of active substance to polymer may be in a range of from about 3:1 to about 1:20. However, narrower ranger of from about 3:1 to about 1:5, such as, e.g., from about 1:1 to about 1:3 or about may also be used.

The solid dispersion is preferably formed by spray drying techniques, controlled agglomeration, freeze-drying or coating on carrier particles or any other solvent removal process. The dried product contains the active substance present in the form of a solid dispersion including a molecular dispersion and a solid solution.

As an alternative to the use of organic solvents the drug and polymer may be co-grinded or extruded at elevated temperatures (melt extrusion).

The pharmaceutical compositions comprising danazol at least partly in form of a solid dispersion or solution may in principle be prepared using any suitable procedure for preparing pharmaceutical compositions known within the art.

Apart from using the organic solvent based method, solid dispersion or solid solutions of danazol and/or an analogue thereof may be obtained by dispersing and/or dissolving danazol in the carrier composition used in the controlled agglomeration method. Stabilizing agents etc. may be added in order to ensure the stability of the solid dispersion/solution.

In another aspect, the invention relates to a method for the preparation of a pharmaceutical composition according to the invention. In general, any suitable method within the pharmaceutical field may be employed. However, in order to enable incorporation of a relatively high amount of an oily material especially the method described in WO 03/004001 (by the same inventors) has proved satisfactory. Details concerning the method are given in the above-identified publication, which is hereby incorporated by reference as well as in the Examples herein. In short, the invention provide a process for preparing a particulate pharmaceutical material comprising danazol and/or an analogue thereof which method comprises spraying a first composition in liquid form, said composition comprising a carrier and having a melting point greater than 5° C. onto a second composition comprising a support, said second composition being in the fluidised state and having a temperature less than the melting point of the carrier. In principle the active substance may be present in the carrier composition and/or in the second composition. However, in those cases where danazol and/or an analogue thereof should be present in the composition at least partly as a solid dispersion, it is advantageous to incorporate or dissolve danazol and/or an analogue thereof in the carrier composition.

Solid Dosage Forms

A pharmaceutical composition according to the invention is in particulate form and may be employed as such. However, in many cases it is more convenient to present the composition in the form of granules, pellets, microspheres, nanoparticles and the like or in the form of solid dosage forms including tablets, capsules and sachets and the like.

A solid dosage form according to the invention may be a single unit dosage form or it may in the form of a polydepot dosage form contain a multiplicity of individual units such as, e.g., pellets, beads and/or granules.

Normally, a pharmaceutical composition or a solid dosage form of the invention is intended for administration via the oral, buccal or sublingual administration route.

The invention also relates to the above-mentioned presentation form. Within the scope of the invention are compositions/solid dosage forms that are intended to release danazol and/or an analogue thereof in a fast release, a delayed release or modified release manner.

A solid dosage form according to the present invention comprises a pharmaceutical composition in particulate form as described above. The details and particulars disclosed under this main aspect of the invention apply mutatis mutandis to the other aspects of the invention. Accordingly, the properties with respect to increase in bioavailability, changes in bioavailability parameters, reduction in adverse food effect as well as release of danazol and/or an analogue thereof etc. described and/or claimed herein for pharmaceutical compositions in particulate form are analogues for a solid dosage form according to the present invention.

Normally, the concentration of the pharmaceutical composition in particulate form is in a range of from about 5 to 100% w/w such as, e.g., from about 10% to about 90% w/w, from about 15% to about 85% w/w, from about 20% to about 80% w/w, from about 25% to about 80% w/w, from about 30% to about 80% w/w, from about 35% to about 80% w/w, from about 40% to about 75% w/w, from about 45% to about 75% w/w or from about 50% to about 70% w/w of the dosage form. In an embodiment of the invention, the concentration of the pharmaceutical composition in particulate form is 50% w/w or more of the dosage form.

A solid dosage form according to the invention is obtained by processing the particulate material according to the invention by means of techniques well-known to a person skilled in the art. Normally, it involves further addition of one or more of the pharmaceutically acceptable excipients mentioned herein.

The composition or solid dosage form according to the invention may be designed to release danazol and/or a derivative and/or an analogue thereof in any suitable manner provided that the increase in bioavailability is present. Thus, the active substance may be released relatively fast in order to obtain an enhanced on-set of action, it may be released so as to follow zero or first order kinetics or it may be released in a controlled or modified manner in order to obtain a predetermined pattern of release. Plain formulations are also within the scope of the present invention.

The composition or solid dosage form according to the invention may also be coated with a film coating, an enteric coating, a modified release coating, a protective coating, an anti-adhesive coating etc.

A solid dosage form according to the invention may also be coated in order to obtain suitable properties e.g. with respect to release of the active substance.

The coating may be applied on single unit dosage forms (e.g. tablets, capsules) or it may be applied on a polydepot dosage form or on its individual units.

Suitable coating materials are e.g. methylcellulose, hydroxypropylmethylcellulose, hydroxypropylcellulose, acrylic polymers, ethylcellulose, cellulose acetate phthalate, polyvinyl acetate phthalate, hydroxypropyl methylcellulose phthalate, polyvinylalcohol, sodium carboxymethylcellulose, cellulose acetate, cellulose acetate phthalate, gelatin, methacrylic acid copolymer, polyethylene glycol, shellac, sucrose, titanium dioxide, carnauba wax, microcrystalline wax, zein.

Plasticizers and other ingredients may be added in the coating material. The same or different active substance may also be added in the coating material.

In the following is given a more detailed description of interesting embodiments of the invention, i.e. embodiments wherein the solid dosage forms are designed to release danazol and/or an analogue thereof in a modified or delayed manner. In the present context, the term “modified release” is intended to include all types of release which differ from the release obtained from plain tablets. Thus, the term includes so-called “controlled release”, “sustained release”, “pulsed release”, “prolonged release”, burst release”, “slow release”, “extended release”, as well as the term “delayed release”. However, a specific aspect of the invention relates to a delayed release composition or dosage form, which in this context is intended to denote a composition or dosage form that at the most releases 10% w/w of the active substance within the first 2 hours after administration and/or after start of a dissolution test employing a dissolution medium having a pH of at the most about 3.

Types of Modified Release Systems

A first class includes matrix systems, in which danazol is embedded or dispersed in a matrix of another material that serves to retard the release of danazol into an aqueous environment (i.e., the luminal fluid of the GI tract). When danazol is dispersed in a matrix of this sort, release of the drug takes place principally from the surface of the matrix. Thus the drug is released from the surface of a device, which incorporates the matrix after it diffuses through the matrix or when the surface of the device erodes, exposing the drug. In some embodiments, both mechanisms can operate simultaneously. The matrix systems may be large, i.e., tablet sized (about 1 cm), or small (<0.3 cm). The system may be unitary (e.g., a bolus), may be divided by virtue of being composed of several sub-units (for example, several capsules which constitute a single dose) which are administered substantially simultaneously, or may comprise a plurality of particles, also denoted a multiparticulate. A multiparticulate can have numerous formulation applications. For example, a multiparticulate may be used as a powder for filling a capsule shell, or used per se for mixing with food to increase palatability.

In a specific embodiment, a matrix multiparticulate, comprises a plurality of danazol-containing particles, each particle comprising danazol and/or an analogue thereof e.g. in the form of a solid dispersion with one or more excipients selected to form a matrix capable of controlling the dissolution rate of the danazol into an aqueous medium. The matrix materials useful for this embodiment are generally water-insoluble materials such as waxes, cellulose, or other water-insoluble polymers. If needed, the matrix materials may optionally be formulated with water-soluble materials, which can be used as binders or as enhancers. Matrix materials useful for the manufacture of these dosage forms such as: Hydroxypropyl methyl cellulose, waxes such as paraffin, modified vegetable oils, carnauba wax, hydrogenated castor oil, beeswax, and the like, as well as synthetic polymers such as poly(vinyl chloride), poly(vinyl acetate), copolymers of vinyl acetate and ethylene, polystyrene, and the like. Water soluble binders or release modifying agents which can optionally be formulated into the matrix include water-soluble polymers such as hydroxypropyl cellulose (HPC), hydroxypropyl methyl cellulose (HPMC), methyl cellulose, poly(N-vinyl-2-pyrrolidinone) (PVP), poly(ethylene oxide) (PEO), poly(vinyl alcohol) (PVA), xanthan gum, carrageenan, and other such natural and synthetic materials. In addition, materials, which function as release-modifying agents include water-soluble materials such as sugars or salts. Preferred water-soluble materials include lactose, sucrose, glucose, and mannitol, as well as HPC, HPMC, and PVP.

In a specific embodiment, a multiparticulate product is defined as being processed by controlled agglomeration. In this case danazol is dispersed in a suitable meltable carrier and sprayed on carrier particles comprising the matrix substance. Alternatively, danazol is dispersed in an organic solvent together with the matrix substance and spray dried or applied to carrier particles.

Solvents typically employed for the process include acetone, ethanol, isopropanol, ethyl acetate, and mixtures of two or more (for further details reference is given to the paragraphs under the heading Description of a solid dispersion based on organic solvents).

Once formed, danazol matrix multiparticulates may be blended with compressible excipients such as lactose, microcrystalline cellulose, dicalcium phosphate, and the like and the blend compressed to form a tablet. Disintegrants such as sodium starch glycolate or crosslinked poly(vinyl pyrrolidone) are also usefully employed. Tablets prepared by this method disintegrate when placed in an aqueous medium (such as the GI tract), thereby exposing the multiparticulate matrix, which releases danazol therefrom.

A further embodiment of a matrix system has the form of a hydrophilic matrix tablet containing danazol and/or an analogue thereof (e.g. in the form of a solid dispersion) as a multiparticulate product and an amount of hydrophilic polymer sufficient to provide a useful degree of control over the danazol dissolution. Hydrophilic polymers useful for forming the matrix include hydroxypropylmethyl cellulose (HPMC), hydroxypropyl cellulose (HPC), poly(ethylene oxide), poly(vinyl alcohol), xanthan gum, carbomer, carrageenan, and zooglan. A preferred material is HPMC. Other similar hydrophilic polymers may also be employed. In use, the hydrophilic material is swollen by, and eventually dissolves in, water. The danazol is released both by diffusion from the matrix and by erosion of the matrix. The danazol dissolution rate of these hydrophilic matrix tablets may be controlled by the amount and molecular weight of hydrophilic polymer employed. In general, using a greater amount of the hydrophilic polymer decreases the dissolution rate, as does using a higher molecular weight polymer. Using a lower molecular weight polymer increases the dissolution rate. The dissolution rate may also be controlled by the use of water-soluble additives such as sugars, salts, or soluble polymers. Examples of these additives are sugars such as lactose, sucrose, or mannitol, salts such as NaCl, KCl, NaHCO₃, and water soluble polymers such as PNVP or PVP, low molecular weight HPC or HMPC or methyl cellulose. In general, increasing the fraction of soluble material in the formulation increases the release rate. A matrix tablet typically comprises about 20 to 90% by weight of danazol and about 80 to 10% by weight of polymer.

A preferred matrix tablet comprises, by weight, about 30% to about 80% solid dispersion containing danazol and/or an analogue thereof about 15% to about 35% matrix former (such as, e.g., HPMC), 0% to about 35% lactose, 0% to about 20% microcrystalline cellulose, and about 0.25% to about 2% lubricant (such as, e.g., magnesium stearate).

The matrix systems as a class often exhibit non-constant release of the drug from the matrix. This result may be a consequence of the diffusive mechanism of drug release, and modifications to the geometry of the dosage form can be used to advantage to make the release rate of the drug more constant.

A second class of danazol sustained-release dosage forms of this invention includes membrane-moderated or reservoir systems. In this class, a reservoir of danazol e.g. in a solid dispersion as a multiparticulate product is surrounded by a rate-limiting membrane. The danazol traverses the membrane by mass transport mechanisms well known in the art, including but not limited to dissolution in the membrane followed by diffusion across the membrane or diffusion through liquid-filled pores within the membrane. These individual reservoir system dosage forms may be large, as in the case of a tablet containing a single large reservoir, or multiparticulate, as in the case of a capsule or poly-depot tablets containing a plurality of reservoir particles, each individually coated with a membrane. The coating can be non-porous, yet permeable to danazol (for example danazol may diffuse directly through the membrane), or it may be porous. As with other embodiments of this invention, the particular mechanism of transport is not believed to be critical.

Sustained release coatings as known in the art may be employed to fabricate the membrane, especially polymer coatings, such as a cellulose ester or ether, an acrylic polymer, or a mixture of polymers. Preferred materials include ethyl cellulose, cellulose acetate and cellulose acetate butyrate. The polymer may be applied as a solution in an organic solvent or as an aqueous dispersion or latex. The coating operation may be conducted in standard equipment such as a fluid bed coater, a Wurster coater, or a rotary fluid bed coater.

If desired, the permeability of the coating may be adjusted by blending of two or more materials. A particularly useful process for tailoring the porosity of the coating comprises adding a pre-determined amount of a finely-divided water-soluble material, such as sugars or salts or water-soluble polymers to a solution or dispersion (e.g., an aqueous latex) of the membrane-forming polymer to be used. When the dosage form is ingested into the aqueous medium of the GI tract, these water soluble membrane additives are leached out of the membrane, leaving pores which facilitate release of the drug. The membrane coating can also be modified by the addition of plasticizers, as known in the art.

A particularly useful variation of the process for applying a membrane coating comprises dissolving the coating polymer in a mixture of solvents chosen such that as the coating dries, a phase inversion takes place in the applied coating solution, resulting in a membrane with a porous structure.

In general, a support for mechanically strengthening the membrane is not required.

The morphology of the membrane is not of critical importance so long as the permeability characteristics enumerated herein are met. The membrane can be amorphous or crystalline. It can have any category of morphology produced by any particular process and can be, for example, an interfacially-polymerized membrane (which comprises a thin rate-limiting skin on a porous support), a porous hydrophilic membrane, a porous hydrophobic membrane, a hydrogel membrane, an ionic membrane, and other such materials which are characterized by controlled permeability to danazol.

A sustained release coating as known in the art, especially polymer coatings, may be employed to fabricate the membrane. Suitable and preferred polymer coating materials, equipment, and coating methods also include those previously discussed.

The rate of danazol release from the coated multiparticulates can also be controlled by factors such as the composition and binder content of the drug-containing core, the thickness and permeability of the coating, and the surface-to-volume ratio of the multiparticulates. It will be appreciated by those skilled in the art that increasing the thickness of the coating will decrease the release rate, whereas increasing the permeability of the coating or the surface-to-volume ratio of the multiparticulates will increase the release rate. If desired, the permeability of the coating may be adjusted by blending of two or more materials. A useful series of coatings comprises mixtures of water-insoluble and water-soluble polymers, for example, ethylcellulose and hydroxypropyl methylcellulose, respectively. A particularly useful modification to the coating is the addition of finely-divided water-soluble material, such as sugars or salts. When placed in an aqueous medium, these water soluble membrane additives are leached out of the membrane, leaving pores which facilitate delivery of the drug. The membrane coating may also be modified by the addition of plasticizers, as is known to those skilled in the art.

In one embodiment of the invention it is an aim to reduce the exposure of the upper GI tract to high concentrations of danazol. Accordingly, suitable dosage forms include those forms, which incorporate a delay before the onset of sustained release of danazol. An exemplary embodiment can be illustrated by a tablet (or a particulate material) comprising a core containing danazol coated with a first coating of a polymeric material of the type useful for sustained release of danazol and a second coating of the type useful for delaying release of drugs when the dosage form is ingested. The first coating is applied over and surrounds the tablet or individual particles. The second coating is applied over and surrounds the first coating.

A tablet can be prepared by techniques well known in the art and contains a therapeutically useful amount of danazol plus such excipients as are necessary to form the tablet by such techniques.

The first coating may be a sustained release coating as known in the art, especially polymer coatings, to fabricate the membrane, as previously discussed for reservoir systems. Suitable and preferred polymer coating materials, equipment, and coating methods also include those previously discussed.

Materials useful for preparing the second coating on the tablet include polymers known in the art as enteric coatings for delayed-release of pharmaceuticals. These most commonly are pH-sensitive materials such as cellulose acetate phthalate, cellulose acetate trimellitate, hydroxypropyl methyl cellulose phthalate, poly(vinyl acetate phthalate), and acrylic copolymers such as Eudragit L-100 (Röhm Pharma) and related materials, as more fully detailed below under “Delayed Release”. The thickness of the delayed-release coating is adjusted to give the desired delay property. In general, thicker coatings are more resistant to erosion and, consequently, yield a longer delay. Preferred coatings range from about 300 μm in thickness to about 3 mm in thickness.

When ingested, the twice-coated tablet passes through the stomach, where the second coating prevents release of the danazol under the acidic conditions prevalent there. When the tablet passes out of the stomach and into the small intestine, where the pH is higher, the second coating erodes or dissolves according to the physicochemical properties of the chosen material. Upon erosion or dissolution of the second coating, the first coating prevents immediate or rapid release of the danazol and modulates the release so as to prevent the production of high concentrations, thereby minimizing side-effects.

A further preferred embodiment comprises a multiparticulate wherein each particle is dual coated as described above for tablets, first with a polymer designed to yield sustained release of the danazol and then coated with a polymer designed to delay onset of release in the environment of the GI tract when the dosage form is ingested.

The sustained release coating may be as known in the art, especially polymer coatings, to fabricate the membrane, as previously discussed for reservoir systems. Suitable and preferred polymer coating materials, equipment, and coating methods also include those previously discussed.

The rate of danazol release from the sustained-release-coated multiparticulates (i.e., the multiparticulates before they receive the delayed-release coating) and methods of modifying the coating are also controlled by the factors previously discussed for reservoir system danazol multiparticulates.

The second membrane or coating for dual coated multiparticulates is a delayed-release coating which is applied over the first sustained-release coating, as disclosed above for tablets, and may be formed from the same materials. It should be noted that the use of the so-called “enteric” materials to practice this embodiment differs significantly from their use to produce conventional enteric dosage forms. With conventional enteric forms, the object is to delay release of the drug until the dosage form has passed the stomach and then to deliver the dose in the duodenum. Dosing of danazol directly and completely to the duodenum may be undesirable, however, due to the side effects sought to be minimized or avoided by this invention. Therefore, if conventional enteric polymers are to be used to practice this embodiment, it may be necessary to apply them significantly more thickly than in conventional practice, in order to delay drug release until the dosage form reaches the lower GI tract. However, it is also possible to effect a sustained or controlled delivery of danazol after the delayed-release coating has dissolved or eroded, therefore the benefits of this embodiment may be realized with a proper combination of delayed-release character with sustained-release character, and the delayed-release part alone may or may not necessarily conform to USP enteric criteria. The thickness of the delayed-release coating is adjusted to give the desired delay property. In general, thicker coatings are more resistant to erosion and, consequently, yield a longer delay.

A first delayed release embodiment according to the invention is a “pH-dependent coated tablet”, which comprises a tablet core comprising danazol e.g. in a solid dispersion as a multiparticulate product, a disintegrant, a lubricant, and one or more pharmaceutical carriers, such core being coated with a material, preferably a polymer, which is substantially insoluble and impermeable at the pH of the stomach, and which is more soluble and permeable at the pH of the small intestine. Preferably, the coating polymer is substantially insoluble and impermeable at pH<5.0, and water-soluble at pH>5.0. The tablet core may be coated with an amount of polymer sufficient to assure that substantially no release of danazol from the dosage form occurs until the dosage form has exited the stomach and has resided in the small intestine for about 15 minutes or greater, preferably about 30 minutes or greater, thus assuring that minimal danazol is released in the duodenum. Mixtures of a pH-sensitive polymer with a water-insoluble polymer may also be employed. Tablets are coated with an amount of polymer comprising from about 10% to about 80% of the weight of the danazol-containing tablet core. Preferred tablets are coated with an amount of polymer comprising about 15% to about 50% of the weight of the danazol tablet core.

pH-sensitive polymers which are relatively insoluble and impermeable at the pH of the stomach, but which are more soluble and permeable at the pH of the small intestine and colon include polyacrylamides, phthalate derivatives such as acid phthalates of carbohydrates, amylose acetate phthalate, cellulose acetate phthalate, other cellulose ester phthalates, cellulose ether phthalates, hydroxypropylcellulose phthalate, hydroxypropylethylcellulose phthalate, hydroxypropylmethylcellulose phthalate, methylcellulose phthalate, polyvinyl acetate phthalate, polyvinyl acetate hydrogen phthalate, sodium cellulose acetate phthalate, starch acid phthalate, styrene-maleic acid dibutyl phthalate copolymer, styrene-maleic acid polyvinylacetate phthalate copolymer, styrene and maleic acid copolymers, polyacrylic acid derivatives such as acrylic acid and acrylic ester copolymers, polymethacrylic acid and esters thereof, poly acrylic methacrylic acid copolymers, shellac, and vinyl acetate and crotonic acid copolymers.

Preferred pH-sensitive polymers include shellac; phthalate derivatives, particularly cellulose acetate phthalate, polyvinylacetate phthalate, and hydroxypropylmethylcellulose phthalate; polyacrylic acid derivatives, particularly polymethyl methacrylate blended with acrylic acid and acrylic ester copolymers; and vinyl acetate and crotonic acid copolymers.

Cellulose acetate phthalate (CAP) may be applied to danazol tablets to provide delayed release of danazol until the danazol-containing tablet has passed the sensitive duodenal region, that is to delay the release of danazol in the gastrointestinal tract until about 15 minutes, and preferably about 30 minutes, after the danazol-containing tablet has passed from the stomach to the duodenum. The CAP coating solution may also contain one or more plasticizers, such as diethyl phthalate, polyethyleneglycol-400, triacetin, triacetin citrate, propylene glycol, and others as known in the art. Preferred plasticizers are diethyl phthalate and triacetin. The CAP coating formulation may also contain one or more emulsifiers, such as polysorbate-80.

Anionic acrylic copolymers of methacrylic acid and methylmethacrylate are also particularly useful coating materials for delaying the release of danazol from danazol-containing tablets until the tablets have moved to a position in the small intestine, which is distal to the duodenum. Copolymers of this type are available from RöhmPharma Corp, under the tradenames Eudragit-L® and Eudragit-S®. Eudragit-L® and Eudragit-Se are anionic copolymers of methacrylic acid and methylmethacrylate. The ratio of free carboxyl groups to the esters is approximately 1:1 in Eudragit-L® and approximately 1:2 in Eudragit-S®. Mixtures of Eudragit-L® and Eudragit-S® may also be used. For coating of danazol-containing tablets, these acrylic coating polymers must be dissolved in an organic solvent or mixture of organic solvents. Useful solvents for this purpose are acetone, isopropyl alcohol, and methylene chloride. It is generally advisable to include 5-20% placticizer in coating formulations of acrylic copolymers. Useful plasticizers are polyethylene glycols, propylene glycols, diethyl phthalate, dibutyl phthalate, castor oil, and triacetin.

The delay time before release of danazol, after the “pH-dependent coated tablet” dosage form has exited the stomach, may be controlled by choice of the relative amounts of Eudragit-L® and Eudragit-S® in the coating, and by choice of the coating thickness. Eudragit-L® films dissolve above pH 6.0, and Eudragit-S® films dissolve above 7.0, and mixtures dissolve at intermediate pH's. Since the pH of the duodenum is approximately 6.0 and the pH of the colon is approximately 7.0, coatings composed of mixtures of Eudragit-L® and Eudragit-S® provide protection of the duodenum from danazol. If it is desired to delay release of danazol until the danazol-containing “pH-dependent coated tablet” has reached the colon, Eudragit-S® may be used as the coating material, as described by Dew et al (Br. J. Clin. Pharmac. 14 (1982) 405-408). In order to delay the release of danazol for about 15 minutes or more, preferably 30 minutes or more, after the dosage form has exited the stomach, preferred coatings comprise from about 9:1 to about 1:9 Eudragit-L®/Eudragit-S®, more preferably from about 9:1 to about 1:4 Eudragit-L®/Eudragit-S®. The coating may comprise from about 3% to about 70% of the weight of the uncoated tablet core. Preferably, the coating comprises from about 5% to about 50% of the weight of the tablet core.

The invention is further illustrated in the following examples without limiting it thereto.

Methods

Determination of Weight Variation

The tablets prepared in the Examples herein were subject to a test for weight variation performed in accordance with Ph. Eur.

Determination of Average Tablet Hardness

The tablets prepared in the Examples herein were subject to at test for tablet hardness employing Schleuniger Model 6D apparatus and performed in accordance with the general instructions for the apparatus.

Determination of Disintegration Time

The time for a tablet to disintegrate, i.e. to decompose into particles or agglomerates, was determined in accordance with Ph. Eur.

Determination of Geometric Weight Mean Diameter d_{gw}

The geometric weight mean diameter was determined by employment of a method of laser diffraction dispersing the particulate material obtained (or the starting material) in air. The measurements were performed at 1 bar dispersive pressure in Sympatec Helos equipment, which records the distribution of the equivalent spherical diameter. This distribution is fitted to a log normal volume-size distribution.

When used herein, “geometric weight mean diameter” means the mean diameter of the log normal volume-size distribution.

Determination of Dissolution Rate

The dissolution rate was determined by employment of USP paddle dissolution method at 37° C.

### EXAMPLES

For the preparation of a pharmaceutical composition in particulate form according to the invention the method described in WO 03/004001 (by the present inventors) has been employed. The method ensures a controlled agglomeration process, i.e. a strict control of the growth in particle size while at the same time it is possible to use a relatively large amount of oily material.

### Example 1

Multiparticulate modified release formulation based on coating

| Substance | % |
|---|---|
| Danazol | 2.00 |
| PEG 6000 | 34.65 |
| Poloxamer | 14.85 |
| Lactose 200 Mesh | 48.50 |
| Total | 100.00 |

4.0% w/w danazol was dissolved in a melted mixture of polyethyleneglycol 6000 and Poloxamer 188 (70:30) at 90° C. 318 g of the solid dispersion was sprayed on 300 g of lactose in a fluid bed Strea-1. The granular product was sieved through sieve 0.7 mm and subsequently coated with a semipermeable membrane and filled into hard gelatine capsules.

Different types of coatings were applied. The details follow as Examples 1a, 1b and 1c.

### Example 1a

### Surelease® Coating of CA (Controlled Agglomeration) Generated Particles

250 g of granules prepared as described above is coated with a Surelease® coating by applying 1 kg of the following coating mixture per 250 g granules. The coating mixture is prepared by diluting Surelease® to 12.5% w/w with water. The coating mixture is applied on the granules by means of the same apparatus used for making the granules, the Strea 1 equiped with a Wurster insert using the following conditions:

| | |
|---|---|
| Nozzle position: | bottom |
| Inlet air temperature: | 75-80° C. |
| Product temperature: | approx. 28° C. |
| Nozzle pressure: | 2.5-3.0 bar |
| Spraying rate: | 12 g/min |
| Fluidized air velocity: | 20-25 m³/hour |

In order to obtain a film thickness of about 10 μm, an amount of polymer corresponding to about 57% of the weight of the granules should be employed.

In the same manner as described above, coated granules were prepared by use of various amounts of coating mixture in order to obtain granules having various amounts of film coating applied (i.e. 2%, 10%, 20%, 30%, 40%, and 50% w/w, respectively). In order to obtain a coating of 50% w/w, 1 kg of Surelease® diluted to 12.5% w/w was employed per 250 g granules. The thus coated granules were subjected to a dissolution test in order to test the release rate of Danazole versus the thickness of the film.

Dissolution Test

The coated Danazole granules were subjected to a dissolution test employing in each of the six vessels a dose corresponding to 100 mg of danazole of the granules and 900 ml of phosphate buffer solution pH 7.5, USP as dissolution medium. A Sotax USP apparatus was employed. The dissolution test was performed in accordance with USP, method 2 (paddle-method) and 50 rpm using a phosphate buffer solution, pH 7.5 (USP) as dissolution medium and a temperature of 37° C. In some cases the dissolution medium was 0.1 N hydrochloric acid during the first 2 hours of testing; then the medium was adjusted to pH 6.8 by addition of Na₃PO₄.

900 ml of dissolution medium was placed into each of the 6 vessels of the Sotax apparatus employed. The temperature was controlled thermostatically at 37° C.±0.5° C. In those cases where the sample under testing was a tablet, one tablet was placed into each vessel and the test was started. In those cases where the sample under testing was a sample of a particulate formulation according to the invention, an accurately weighted amount corresponding to one dose of the active substance was placed in each vessel and the test was started. At appropriate intervals a 10 ml sample was removed from each vessel for individual measurement (and replaced with another 10 ml of dissolution medium). The samples were filtered and cooled to room temperature and analyzed.

The following results were obtained (the values given are the mean values of two determinations and the values are given as the weight percentages released after the stated time period):

TABLE 1

| | |
|---|---|
| time | % film coating |

| (hours) | 2% | 10% | 20% | 30% | 40% | 50% |
|---|---|---|---|---|---|---|
| 0.5 | 94.2 | 81.9 | 36.3 | 12.6 | 8.5 | 9.6 |
| 1.0 | 96.3 | 94 | 57.1 | 20.3 | 13.7 | 13.7 |
| 2.0 | 97.2 | 97.2 | 83.7 | 36.7 | 23.5 | 21.9 |
| 3.0 | 101 | 101 | 97.2 | 51.9 | 35.5 | 31.8 |
| 4.0 | 99.3 | 101 | 101 | 63.7 | 45 | 41.6 |
| 5.0 | | 99.7 | 102 | 76.3 | 56 | 48.6 |
| 6.0 | | 99.8 | 102 | 86.2 | 63.8 | 57.5 |
| 24 | | 99.9 | 104 | 106 | 101 | 98.4 |

The results clearly show that the granules prepared by controlled agglomeration are sufficiently robust to withstand a coating procedure and that a modified release coating can be made. Furthermore, the results show that the retardation in release increases as the coating thickness increases. Granules coated with 2% w/w or 10% w/w Surelease® release almost instantly the total amount of danazole contained in the granules

### Example 1b

### Ethyl Cellulose Coating of Granules Prepared by Controlled Agglomeration Technique

250 g of granules prepared as described above are coated with an ethyl cellulose coating by applying 625 g of the following coating mixture per 250 g granules. The coating mixture is prepared by dissolving 10% of ethylcellulose 20 cps in ethanol and adding 8% w/w DBS (dibutylsebacate) as a plasticizer (625 g coating solution per 250 g granules have the following composition:

| | |
|---|---|
| Ethanol | 560 g |
| Ethocel ® | 60 g |
| Dibuthylsebacate | 5 g |

corresponding to 9.9% w/w Ethocel® as dry matter and 0.8% w/w dibuthylsebacetate as dry matter).

The coating solution is applied on the granules by means of the controlled agglomeration apparatus (Strea 1′ equipped with a Wurster insert) using the following conditions:

| | |
|---|---|
| Nozzle position: | bottom |
| Inlet air temperature: | 50-65° C. |
| Product temperature: | 28-35° C. |
| Nozzle pressure: | 3.0 bar |
| Spraying rate: | 15 g/min. |
| Fluidized air velocity: | 20-22 m³/hour |

A film coating having a thickness of about 5 μm is obtained. 625 kg coating solution per 250 g granules is applied, corresponding to 112.5 g dry matter per 250 g granules (45% w/w).

In the same manner as described above, coated granules were prepared by use of various amounts of coating mixture in order to obtain granules having various amounts of film coating applied (i.e. 8.6%, 11.9%, 16.2%, 20.5%, 24.8%, and 27% w/w, respectively). The thus coated granules were subjected to a dissolution test in order to test the release rate of Danazole versus the thickness of the film.

Dissolution Test

The coated Danazole granules were subjected to a dissolution test employing in each of the vessels 100 mg of danazole of the granules and 900 ml of phosphate buffer solution pH 7.5, USP as dissolution medium (see above for details).

The following results were obtained (the values given are the mean values of two determinations and the values given are the weight percentages released after the stated time period):

TABLE 2

| time | % film coating | | | | | |
|---|---|---|---|---|---|---|
| (hours) | 8.6% | 11.9% | 16.2% | 20.5% | 24.8% | 27% |
| 0.5 | 34.3 | 34.5 | 7.1 | 3.1 | 2.6 | 1.1 |
| 1.0 | 48.7 | 42.7 | 12.4 | 4.8 | 3.5 | 1.9 |
| 2.0 | 63.7 | 55.4 | 20.1 | 8.8 | 6.8 | 4.3 |
| 3.0 | 73 | 69.2 | 27.2 | 11.7 | 9.3 | 6.4 |
| 4.0 | 76.2 | 70.9 | 29.6 | 12.7 | 10.1 | 7 |
| 5.0 | 79.7 | 73.1 | 33.5 | 14.2 | 11.8 | 8.7 |
| 6.0 | 80.3 | 74.5 | 36.8 | 16.4 | 13.7 | 10.6 |
| 24 | 98.7 | 83.5 | 62.1 | 36.2 | 26 | 21 |

The results clearly show that the granules prepared by controlled agglomeration are sufficiently robust to withstand a coating procedure and that a modified release coating can be obtained. Furthermore, the results show that the retardation in release increases as the coating thickness increases. Granules coated with 8.6% w/w or 1.1.9% w/w ethylcellulose display also a modified release pattern. The results also show that less film is needed when using an ethanol-based film than when an aqueous based film is used. This is most likely due to the dissolution characteristics of ethylcellulose in ethanol as ethylcellulose easily dissolves in ethanol and thus perform a more homogeneous and tight film on the granules. In the case of an aqueous based film, the polymer (i.e. ethyl-cellulose) is dispersed in the medium as small particles (dispersion), which makes the coating more difficult.

### Example 1c

### Eudragit® Coating of Granules

0.25 kg of granules prepared as described above is coated with 0.610 kg of the following coating mixture containing Eudragit® RS 30D as a 30% w/w dispersion in water:

| | |
|---|---|
| Eudragit ® RS 30D (30% w/w dispersion) | 475.0 g |
| (corresponding to 142.5 g dry matter) | |
| Triethyl citrate (Eudraflex ®) | 28.5 g |
| Microtalcum | 71.3 g |
| Antifoam M 10 | 3.0 g |
| Purified water | 640.0 g |

The coating mixture is applied on the granules by means of a controlled agglomeration apparatus (Strea 1 equipped with a Wurster insert) using the following conditions:

| | |
|---|---|
| Nozzle position: | bottom |
| Nozzle size | 0.8 mm |
| Inlet air temperature: | 60° C.-75° C. |
| Product temperature: | 25° C.-34° C. |
| Nozzle pressure: | 2 bar |
| Spraying rate: | up to 9 g/min |
| Fluidized air velocity: | up to 25 m³/hour |

A film coating having a thickness of about 10 μm is obtained. About 43% w/w dry matter is applied on the granules. The thus coated granules were subjected to a dissolution test in order to test the release rate of danazole versus time.

Dissolution Test

The coated danazole granules were subjected to a dissolution test employing in each of the vessels a dose corresponding to 300 mg of danazole of the granules and 900 ml of 0.1 N hydrochloric acid as dissolution medium. After 2 hours the pH of the dissolution medium was adjusted to pH 6.8 by addition of Na₃PO₄ (see above).

The following results were obtained (the values given are the mean values of two determinations and the values given are weight percentages released after the stated time period):

TABLE 4

| | % film coating |
|---|---|
| time (hours) | 43% |
| 0.5 | 6.1 |
| 1.0 | 9.8 |
| 2.0 | 17.8 |
| 3.0 | 21.0 |
| 4.0 | 27.1 |
| 5.0 | 31.3 |
| 6.0 | 35.3 |
| 24 | 80.8 |

Alternatively granule products might be compressed into a tablet followed by coating of the tablet with a film membrane or compression coating of the tablet core.

### Example 2

Tablet modified release formulation based on coating

| Substance | % | mg |
|---|---|---|
| Danazol | 2.00 | 5.00 |
| PEG 6000 | 34.65 | 86.63 |
| Poloxamer 188 | 14.85 | 37.13 |
| Lactose 200 Mesh | 47.50 | 118.75 |
| Magnesium stearate | 1.00 | 2.50 |
| Total | 100.00 | 250.00 |

4.0% Danazol was dissolved in a melted mixture of polyethyleneglycol 6000 and Poloxamer 188 (70:30) at 90° C. 318 g of the solid dispersion was sprayed on 300 g of lactose in a fluid bed Strea-1. The granular product was sieved through sieve 0.7 mm and mixed with 1% magnesium stearate for 0.5 min in a Turbula mixer. 8 mm tablets (compound cup) were compressed on a Korsch EKO with a weight of 250 mg and strength 5 mg. Mean tablet hardness: 75 N. The tablets were subsequently coated with a semipermeable membrane as in Example 1a (Surelease coating).

### Example 3

### Delayed Release Tablet Formulation

Tablet Composition:

| Substance | % | mg |
|---|---|---|
| Danazol | 2.00 | 5.00 |
| PEG 6000 | 34.65 | 86.63 |
| Poloxamer 188 | 14.85 | 37.13 |
| Lactose 200 Mesh | 47.50 | 118.75 |
| Magnesium stearate | 1.00 | 2.50 |
| Total | 100.00 | 250.00 |

4.0% Danazol was dissolved in a melted mixture of polyethyleneglycol 6000 and Poloxamer 188 (70:30) at 90° C. 318 g of the solid dispersion was sprayed on 300 g of lactose in a fluid bed Strea-1. The granular product was sieved through sieve 0.7 mm and mixed with 1% magnesium stearate for 0.5 min in a Turbula mixer. 8 mm tablets (compound cup) were compressed on a Korsch EKO with a weight of 250 mg and strength 5 mg. Mean tablet hardness: 75 N. The tablets were subsequently enteric coated with an aqueous based latex suspension of Eudragit L30D (methacrylic acid co-polymer), Röhm Pharma. The film composition is shown below

Film Composition:

| Substance | % |
|---|---|
| Eudragit L30D | 40.0 |
| Water | 52.0 |
| Triethyl citrate | 1.8 |
| Silicon oil | 0.2 |
| Talc | 6.0 |

400 g tablets were coated in a Strea-1 with a Wurster insert (bottom spray) using the following process conditions: Liquid flow rate: 7 g/min, inlet air temperature 60-65° C. Product temperature: 29-31° C. Outlet air temp: 26-28° C. Inlet air flow: 18-25 m³/hour.

The tablets were coated until a weight gain of 4% was obtained and cured for 24 hours at 30° C.

### Example 4

### In Vitro and In Vivo Behaviour of Compositions According to the Invention

### Example 4a

### Matrix Tablets/Matrix Capsules with Intragranular Hydrocolloid (Batch No. RD1019-4T and Batch No. RD1019-4K, Respectively)

Tablet Composition (Batch No. RD1019-4T):

| Substance | % | mg |
|---|---|---|
| Danazol | 1.91 | 10.05 |
| Metolose HS 90 100 cP | 20.86 | 109.53 |
| Lactose 200 mesh | 31.30 | 164.30 |
| PEG 6000 | 32.15 | 168.78 |
| Poloxamer 188 | 13.78 | 72.33 |
| Total | 100.00 | 525.00 |

4.0% Danazol was dissolved in a melted mixture of polyethyleneglycol 6000 and Poloxamer 188 (70:30) at 90° C. 318 g of the solid dispersion was sprayed on a mixture of 150 g of lactose and 100 g Metolose 90SH 100 cP in a fluid bed Strea-1. The granular product was sieved through sieve 0.7 mm. The product was directly compressed into 12 mm tablets (compound cup) on a Diaf TM20. The tablets had a mean weight of 525 mg and a strength of 10 mg. Mean tablet hardness: 52 N.

Tablets having a strength of 5 mg were also prepared as well as capsules having a strength of 10 mg (i.e. the granular product obtained above was filled into capsules). The two dosage forms were tested in vivo in dogs (see Example 4d).

### Example 4b

### Matrixtablet with Intragranular Hydrocolloid (Batch No. RD1019-3T)

Tablet Composition (Batch No. RD1019-3T):

| Substance | % | mg |
|---|---|---|
| Danazol | 1.91 | 10.05 |
| Metolose HS 90 100,000 Cp | 20.86 | 109.53 |
| Lactose 200 mesh | 31.30 | 164.30 |
| PEG 6000 | 32.15 | 168.78 |
| Poloxamer 188 | 13.78 | 72.33 |
| Total | 100.00 | 525.00 |

The tablets were similar to those of Example 4a apart from the quality of Metolose used.

Tablets containing 5 mg danazol were also tested in dogs. The tablets had the following in vitro dissolution.

100,000 cP, RD1019-3T, 5 mg

| time (min) | dissolved (%) |
|---|---|
| 2 | 1.6 |
| 5 | 2.6 |
| 15 | 3.1 |
| 30 | 3.5 |
| 45 | 4.1 |
| 60 | 4.9 |
| 90 | 7.5 |
| 120 | 10.3 |
| 180 | 35.1 |
| 300 | 86.9 |
| 420 | 99.2 |
| 1200 | 107.0 |

The results obtained from the study in dogs follow in Example 4d

### Example 4c

### Multiparticulate Modified Release Capsule with Intragranular Hydrocolloid (Batch No. RD1019-5K)

Capsule Composition:

| Substance | % | mg |
|---|---|---|
| Danazol | 2.00 | 10.05 |
| Metolose HS 90 100000 cp | 50.00 | 109.53 |
| PEG 6000 | 33.60 | 168.00 |
| Poloxamer 188 | 14.40 | 72.00 |
| Total | 100.00 | 500.00 |

4.0% Danazol was dissolved in a melted mixture of polyethyleneglycol 6000 and Poloxamer 188 (70:30) at 90° C. 200 g of the solid dispersion was sprayed on 200 g Metolose 90SH 100000 cP in a fluid bed Strea-1. The granular product was sieved through sieve 0.7 mm. and filled into hard gelatine capsules (500 mg).

Capsules having a strength of 5 mg were also produced and tested in vivo in dogs (results are given in Example 4d).

### Example 4d

### Preclinical Testing of Compositions According to the Invention in Dogs

The tablets from Examples 4a-4-c were tested in dogs and compared with a commercially available capsule composition, Danocrine, that had the following in vitro dissolution properties:

Danocrine 10 mg

| time (min) | dissolved (%) |
|---|---|
| 2 | 105.9 |
| 5 | 108.6 |
| 15 | 111.3 |
| 30 | 111.3 |
| 45 | 110.9 |
| 60 | 110.9 |
| 420 | 110.8 |

The dogs were given a total dose of 20 mg danazol. The tablets or capsules were administered orally.

The results are given in FIG. 1 and in the following table:

| | | AUC_{0-inf} | Cₘₐₓ | | kₑ | t_{1/2} |
|---|---|---|---|---|---|---|
| Composition | Dose | ng/ml ' h | ng/ml | Tₘₐₓ h | h⁻¹ | h |
| Danocrine | 20 mg | 101.3 | 33.8 | 2 | 0.521 | 1.33 |
| | capsules | | | | | |
| Example 4a | 4 × 5 mg | 312.5 | 75.9 | 1.5 | 0.143 | 4.85 |
| RD1019-4T | tablets | | | | | |
| Example 4a | 2 × 10 mg | 286.6 | 80.3 | 1.5 | 0.127 | 5.46 |
| RD1019-4K | capsules | | | | | |
| Example 4b | 4 × 5 mg | 223.1 | 63.0 | 2 | 0.150 | 4.63 |
| RD1019-3T | tablets | | | | | |
| Example 4c | 4 × 5 mg | 198.8 | 44.0 | 2.3 | 0.306 | 2.26 |
| RD1019-5K | capsules | | | | | |

Compared with Danocrine capsules, the compositions of the invention resulted in an increased bioavailability, an increased Cₘₐₓ, a reduced elimination rate constant and a prolonged T_{1/2}.

### Example 5

### Matrixtablet with Extragranular Hydrocolloid

Tablet Composition:

| Substance | % | mg |
|---|---|---|
| Danazol | 1.61 | 10.00 |
| Lactose 200 mesh | 38.14 | 237.5 |
| PEG 6000 | 27.83 | 173.3 |
| Poloxamer 188 | 11.93 | 74.3 |
| Metolose HS 90 15000 cP | 20.00 | 124.5 |
| Magnesium stearate | 0.5 | 3.1 |
| Total | 100.00 | 623 |

The granular product from Example 1 is mixed with 20% Metolose 90 SH 15000 cP in a turbula mixer for 3 minutes and subsequently mixed with 0.5% magnesium stearate for 0.5 min. The granulate was directly compressed into 12 mm tablets (compound cup) on a Diaf TM20. The tablets had a mean weight of 623 mg and a strength of 10 mg. Mean tablet hardness: 41 N.

1. A composition comprising danazol as an active substance together with one or more pharmaceutically acceptable excipients, wherein the composition upon oral administration to a mammal in need thereof releases the active substance in a controlled manner and exhibits an AUC/AUC_{Control} value of at least about 1.5, the AUC values being determined under similar conditions.

2. The composition according to claim 1, wherein the AUC/AUC_{Control} value is at least about 1.75 or more such as, e.g., about 1.8 or more, about 1.9 or more, about 2.0 or more, about 2.5 or more, about 2.75 or more, about 3.0 or more, about 3.25 or more, about 3.5 or more, about 3.75 or more, about 4.0 or more, about 4.25 or more, about 4.5 or more, or about 4.75 or more, the AUC values being determined under similar conditions.

3. The composition according to claim 1, wherein the AUC/AUC Control value is at least about 5.0 or more, about 6 or more, about 7 or more, about 8 or more, about 9 or more or about 10 or more, the AUC values being determined under similar conditions.

4. The composition comprising danazol as an active substance together with one or more pharmaceutically acceptable excipients, wherein the composition upon oral administration to a mammal in need thereof releases the active substance in a controlled manner and exhibits a W₅₀ that is about 2 hours or more such as, e.g., about 3 hours or more, about 4 hours or more, about 5 hours or more, about 6 hours or more, about 7 hours or more, about 8 hours or more, about 9 hours or more, about 10 hours or more, about 12 hours or more, about 14 hours or more, about 16 hours or more, about 18 hours or more or about 20 hours or more.

5. A composition comprising danazol as an active substance together with one or more pharmaceutically acceptable excipient, wherein the composition upon oral administration to a mammal in need thereof releases the active substance in a controlled manner and exhibits a C_{diff} of 90 or less such as, e.g., about 85 or less, about 80 or less, about 75 or less, about 70 or less, about 65 or less, about 60 or less, about 55 or less, about 50 or less, about 45 or less or about 40 or less, when C_{diff}=[Cₘₐₓ−C (t=7 hours)] and C_{diff} for Danocrine® tablets is set to 100.

6. A composition comprising danazol as an active substance together with one or more pharmaceutically acceptable excipient, wherein the composition upon oral administration to a mammal in need thereof releases the active substance in a controlled manner and does not exhibit a significant adverse food effect as evidenced by a value of (AUC_{fed}/AUC_{fasted}) of at least about 0.85 with a lower 90% confidence limit of at least 0.75.

7. The composition according to claim 6, wherein the value of (AUC_{fed}/AUC_{fasted}) is about 0.9 or more such as, e.g., about 0.95 or more, about 0.97 or more or about 1 or more.

8. A composition comprising danazol as an active substance together or an analogue thereof with one or more pharmaceutically acceptable excipient, wherein the composition upon oral administration to a mammal in need thereof releases danazol or an analogue thereof in a controlled manner and the composition being essentially bioequivalent with Danocrine® or a similar commercially available danazol-containing product when administered in a dosis that is at the about most about 85% w/w of the dose of danazol administered in the form of Danocrine® or a similar commercially available danazol-containing product.

9. The composition according to claim 8, wherein the dose is at the most about 80% w/w such as, e.g., at the most about 75%, at the most about 70% w/w, at the most about 65% w/w, at the most about 60% w/w, at the most about 55% w/w or at the most about 50% w/w of the dose of danazol administered in the form of Danocrine® or a similar commercially available danazol-containing product.

10. The composition according to claim 8, wherein the bioequivalence is determined by means of at least one of the following parameters: tₘₐₓ, cₘₐₓ, AUC₀₋ₜ, AUC_{0-infinity}, W₅₀, W₇₅ and/or MRT.

11. A composition comprising danazol as an active substance together with one or more pharmaceutically acceptable excipients, wherein the composition upon oral administration to a mammal in need thereof releases the active substance in a controlled manner and reduces gastrointestinal side effects compared to those of Danocrine® administered under the same conditions and in a dose that provides an equivalent therapeutic effect.

12. A composition comprising danazol as an active substance together with one or more pharmaceutically acceptable excipients, wherein the composition upon oral administration to a mammal in need thereof releases the active substance in a controlled manner and reduces inter- and/or intra-individual variations compared to those of Danocrine® administered under the same conditions and in a dose that provides an equivalent therapeutic effect.

13. A composition comprising danazol as an active substance together with one or more pharmaceutically acceptable excipients, wherein the composition upon oral administration to a mammal in need thereof in a controlled manner releases at least about 50% w/w of the total amount of the active substance within about 15 hours such as, e.g., within about 12 hours.

14. A composition according to claim 13, wherein the composition upon oral administration to a mammal in need thereof releases at least about 50% w/w of the total amount of the active substance within about 10 hours such as, e.g., within about 8 hours, within about 6 hours, within about 4 hours or within about 3 hours.

15. The composition according to claim 13, wherein the composition upon oral administration to a mammal in need thereof releases at least about 55% w/w such as, e.g., about 60% w/w or more, about 65% w/w or more, about 70% w/w or more, about 75% w/w or more or about 80% w/w or more of the total amount of the active substance within about 15 hours such as, e.g., within about 12 hours, within about 10 hours, within 8 hours or within about 6 hours.

16. The composition according to claim 13, wherein at least about 50% w/w of the total amount of the active substance is released within 15 hours such as, e.g., within about 12 hours, when tested in an in vitro dissolution test and employing a dissolution medium comprising a buffer having pH 7.5.

17. The composition according to claim 13, wherein at least about 50% w/w of the total amount of the active substance is released within about 10 hours such as, e.g., within about 8 hours, within about 6 hours, within about 4 hours, within about 3 hours or within about 2 hours, when tested in an in vitro dissolution test and employing a dissolution medium comprising a buffer having pH 7.5.

18. The composition according to claim 17, wherein at least about 50% w/w of the total amount of the active substance is released within about 1.5 hours such as, e.g., within about 1 hour, within about 0.75 hours, within about 0.5 hours or within about 20 minutes, when tested in an in vitro dissolution test and employing a dissolution medium comprising a buffer having pH 7.5.

19. The composition according to claim 13, wherein at least about 55% w/w such as, e.g., about 60% w/w or more, about 65% w/w or more, about 70% w/w or more, about 75% w/w or more or about 80% w/w or more of the total amount of the active substance thereof is released within about 15 hours such as, e.g., within about 12 hours, within about 10 hours, within 8 hours or within about 6 hours, when tested in an in vitro dissolution test and employing a dissolution medium comprising a buffer having pH 7.5.

20. The composition according to claim 13, wherein at least about 55% w/w such as, e.g., about 60% w/w or more, about 65% w/w or more, about 70% w/w or more, about 75% w/w or more or about 80% w/w or more of the total amount of the active substance is released within about 5 hours such as, e.g., within about 4 hours, within about 3 hours, within about 2 hours, within about 1 hours or within about 30 minutes, when tested in an in vitro dissolution test and employing a dissolution medium comprising a buffer having pH 7.5.

21. The pharmaceutical composition according to claim 16, wherein the in vitro dissolution test is carried out employing USP dissolution test (paddle) and a buffer pH 7.5 containing 0.75% sodium lauryl sulfate as dissolution medium.

22. The composition according to claim 13, wherein at least about 20% w/w such as, e.g., at least about 25% w/w, at least about 30% w/w, at least about 35% w/w or at least about 40% w/w of the total amount of the active substance is released within the first 3 hours such as, e.g., within the first 2 hours or within the first hour when tested in an in vitro dissolution test and employing a dissolution medium comprising a buffer having pH 7.5.

23. A composition comprising danazol as an active substance together with one or more pharmaceutically acceptable excipient, wherein the composition upon oral administration to a mammal in need thereof has a delayed release of the active substance so that at the most about 10% w/w such as, e.g., at the most about 7.5% w/w or at the most about 5% w/w of the total amount of the active substance is released within the first two hours such as, e.g., within the first hour after administration.

24. The composition according to claim 23, wherein at the most about 30% w/w such as, e.g., at the most about 25% w/w, at the most about 20% w/w, at the most about 15% w/w or at the most about 10% w/w of the active substance is released within 2 hours in an in vitro dissolution test employing a dissolution medium having a pH of at the most about 5 such as, e.g. at the most about 4.5, at the most about 4, at the most about 3.5, at the most about 3, at the most about 2 or at the most about 1.5.

25. The composition according to claim 23, wherein at the most about 10% w/w such as, e.g., at the most about 7.5% w/w, at the most about 5% w/w or at the most about 2.5% w/w of the active substance is released within 2 hours in an in vitro dissolution test employing a dissolution medium having a pH of at the most about 5 such as, e.g. at the most about 4.5, at the most about 4, at the most about 3.5, at the most about 3, at the most about 2 or at the most about 1.5.

26. The composition according to claim 23, wherein at the most about 60% w/w such as, e.g., at the most about 50% w/w, at the most about 40% w/w or at the most about 30% w/w of the active substance is released within 15 hours such as, e.g., within about 12 hours, when tested in an in vitro dissolution test employing a dissolution medium having a pH of at the most about 4.5 such as, e.g. at the most about 4.0, at the most about 3.5, at the most about 3, at the most about 2 or at the most about 1.5.

27. The composition according to claim 23, wherein at the most about 40% w/w such as, e.g., at the most about 30% w/w, at the most about 25% w/w or at the most about 20% w/w of the active substance is released within 6 hours when tested in an in vitro dissolution test employing a dissolution medium having a pH of at the most about 4.5 such as, e.g. at the most about 4.0, at the most about 3.5, at the most about 3, at the most about 2 or at the most about 1.5.

28. The composition according to claim 23, wherein at the most about 30% w/w such as, e.g., at the most about 25% w/w, at the most about 20% w/w or at the most about 15% w/w of the active substance is released within 4 hours when tested in an in vitro dissolution test employing a dissolution medium having a pH of at the most about 4.5 such as, e.g. at the most about 4.0, at the most about 3.5, at the most about 3, at the most about 2 or at the most about 1.5.

29. The composition according to claim 1, wherein said composition is in the form of a particulate material that has a geometric weight mean diameter d_{gw} of ≧10 μm such as, e.g. ≧20 μm, from about 20 to about 2000, from about 30 to about 2000, from about 50 to about 2000, from about 60 to about 2000, from about 75 to about 2000 such as, e.g. from about 100 to about 1500 μm, from about 100 to about 1000 μm or from about 100 to about 700 μm, or at the most about 400 μm or at the most 300 μm such as, e.g., from about 50 to about 400 μm such as, e.g., from about 50 to about 350 μm, from about 50 to about 300 μm, from about 50 to about 250 μm or from about 100 to about 300 μm.

30. The composition according to claim 1, wherein the one or more pharmaceutically acceptable excipients is selected from the group consisting of fillers, disintegrants, binders, diluents, lubricants and glidants.

31. The composition according to claim 1 further comprising an pharmaceutically acceptable additive selected from the group consisting of flavoring agents, coloring agents, taste-masking agents, pH-adjusting agents, buffering agents, preservatives, stabilizing agents, anti-oxidants, wetting agents, humidity-adjusting agents, surface-active agents, suspending agents, absorption enhancing agents.

32. The composition according to claim 1 wherein at least one of the one or more pharmaceutically acceptable excipients is selected from the group consisting of silica acid or a derivative or salt thereof including silicates, silicon dioxide and polymers thereof; magnesium aluminosilicate and/or magnesium aluminometasilicate, bentonite, kaolin, magnesium trisilicate, montmorillonite and/or saponite.

33. The composition according to claim 32 wherein the pharmaceutically acceptable excipient a silica acid or a derivative or salt thereof.

34. The composition according to claim 32, wherein the pharmaceutically acceptable excipient is silicon dioxide or a polymer thereof.

35. The composition according to claim 34, wherein the silicon dioxide product has properties corresponding to Zeofree® 5161A, Zeofree® 5162, Zeofree® 5175A, Zeopharm® 80 (available from J. M. Huber, Hamina, Finland), Aeroperl® 300, Sident® 22S, Sipernat®160, Sipernat® 160PQ, Sipernat® 22, Sipernat® 22 LS, Sipernat® 22, Sipernat® 22 LS, Sipernat® 22S, Sipernat® 2200, Sipernat® 310, Sipernat® 320, Sipernat® 320 DS, Sipernat® 325 C, Sipernat® 35, Sipernat® 350, Sipernat® 360, Sipernat® 383 D8, Sipernat® 44, Sipernat® 44MS, Sipernat® 50, Sipernat® 50S, Sipernat® 50 S, Sipernat® 500 LS, or Sipernat® 570.

36. The composition according to claim 1, wherein said composition comprises an oily material.

37. The composition according to claim 36, wherein the concentration of the oily material in the composition is about 5% w/w or more such as, e.g., about 10% w/w or more, about 15% w/w or more, about 20% w/w or more, about 25% w/w or more, about 30% w/w or more, about 35% w/w or more, about 40% w/w or more, about 45% w/w or more, about 50 w/w or more, about 55% w/w or more, about 60% w/w or more, about 65% w/w or more, about 70% w/w or more, about 75% w/w or more, about 80% w/w or more, about 85% w/w or more, about 90% w/w or more or about 95% w/w or more.

38. The composition according to claim 37, wherein the concentration of the oily material is in a range from about 20% to about 80% w/w such as, e.g., from about 25% to about 75% w/w.

39. The composition according to claim 1, wherein at least a part of danazol is present in the form of a solid dispersion including a molecular dispersion and a solid solution.

40. The composition according to claim 39, wherein the solid dispersion is manufactured by dissolving at least a part of danazol in an organic solvent containing a material suitable for forming solid dispersions and subsequent removing the organic solvent e.g. by evaporation.

41. The composition according to claim 40, wherein the material suitable for forming solid dispersions is selected from the group consisting of cellulose derivatives including hydroxypropylmethylcellulose, NaCMC, PVP and PVA.

42. The composition according to claim 1 having an acceptable flowability as determined according to the method described in Ph.Eur. measuring the flow rate of the material out of a funnel with a nozzle diameter of 10.0 mm.

43. Granules, pellets, microspheres and, nanoparticles comprising the composition of claim 1.

44-45. (canceled)

46. The composition according to claim 44 in the form of tablets, capsules or sachets.

47. The composition according to claim 46, wherein said tablets are obtained by direct compression.

48. A solid dosage form comprising the composition according to any of claim 1.

49. The solid dosage form according to claim 48, wherein the concentration of the composition in particulate form is in a range of from about 5% to 100% w/w such as, e.g., from about 10% to about 90% w/w, from about 15% to about 85% w/w, from about 20% to about 80% w/w, from about 25% to about 80% w/w, from about 30% to about 80% w/w, from about 35% to about 80% w/w, from about 40% to about 75% w/w, from about 45% to about 75% w/w or from about 50% to about 70% w/w of the dosage form.

50. The solid dosage form according to claim 48, wherein the concentration of the composition in particulate form is 50% w/w or more of the dosage form.

51. The solid dosage form according to claim 48, wherein the solid dosage form upon oral administration to a mammal in need thereof exhibits an AUC/AUC_{Control} value of at least about 1.5, the AUC values being determined under similar conditions.

52. The solid dosage form according to claim 51, wherein the AUC/AUC_{Control} value is at least about 1.75 or more such as, e.g., about 1.8 or more, about 1.9 or more, about 2.0 or more, about 2.5 or more, about 2.75 or more, about 3.0 or more, about 3.25 or more, about 3.5 or more, about 3.75 or more, about 4.0 or more, about 4.25 or more, about 4.5 or more, about 4.75 or more, about 5.0 or more, about 6 or more, about 7 or more, about 8 or more, about 9 or more or about 10 or more, the AUC values being determined under similar conditions.

53. The solid dosage form according to claim 48, wherein the solid dosage form releases danazol in a controlled manner and does not exhibit a significant adverse food effect as evidenced by a value of (AUC_{fed}/AUC_{fasted}) of at least about 0.85 with a lower 90% confidence limit of at least 0.75.

54. The solid dosage form according to claim 53, wherein the value of (AUC_{fed}/AUC_{fasted}) is about 0.9 or more such as, e.g., about 0.95 or more, about 0.97 or more or about 1 or more.

55. The solid dosage form according to claim 48, wherein the solid dosage form upon oral administration to a mammal in need thereof releases danazol in a controlled manner and the solid dosage form being essentially bioequivalent with Danocrine® or a similar commercially available danazol-containing product when administered in a dose that is at the most about 85% w/w of the dose of danazol administered in the form of Danocrine® or a similar commercially available danazol containing product.

56. The solid dosage form according to claim 55, wherein the dose is at the most about 80% w/w such as, e.g., at the most about 75%, at the most about 70% w/w, at the most about 65% w/w, at the most about 60% w/w, at the most about 55% w/w or at the most about 50% w/w of the dose of the active substance administered in the form of Danocrine® or a similar commercially available danazol-containing product.

57. (canceled)
